(19) United States
(12) Patent Application Publication
(10) Pub. No.: US 2025/0163522 A1
(43) Pub. Date: May 22, 2025

(54) METHODS FOR PRESERVING HEALTHY INTESTINAL MICROBIOME DURING GROWTH PROMOTER ADMINSTRATION

(71) Applicant: BIOMEDIT, LLC, Fishers, IN (US)

(72) Inventors: German PLATA, Fishers, IN (US); Troy B. HAWKINS, Fishers, IN (US); Arvind KUMAR, Fishers, IN (US)

(21) Appl. No.: 18/694,065

(22) PCT Filed: Sep. 22, 2022
(86) PCT No.: PCT/US2022/044403

§ 371 Date: Mar. 21, 2024

(51) Int. Cl. C12Q 1/689; A61K 31/351; A61K 31/715; A61K 38/12

(52) U.S. Cl. CPC C12Q 1/689; A61K 31/351; A61K 31/715; A61K 38/12; C12Q 2600/106; C12Q 2600/156

(57) ABSTRACT

The present invention relates to methods of treating animals with an antimicrobial growth promoter (AGP) to promote a healthy microbiome, the method comprising: a) providing test organisms and a control organism, b) administering a regular regimen of a different AGP to each test organism for about two months, c) collecting DNA from the intestinal content of the test organisms and of the control organism, d) sequencing the DNA of the intestinal contents of the organisms to obtain taxonomic and functional profiles, thereby defining core intestinal microbiomes of the test and control organisms, e) comparing the core intestinal microbiomes of the test organisms and of the control organism, thereby determining which test organism has a healthy microbiome thereby determining which AGP provides the most healthy microbiome, and f) treating the domesticated organism with the AGP which provides the healthy microbiome.

### BACKGROUND OF THE INVENTION

The discovery in the 1930s that feeding of antimicrobial compounds could accelerate animal growth played a significant role in shaping and expanding animal agriculture to its current scale. Intensive animal food production has sustained an increasing global demand for animal protein but has also generated concerns about selection for antimicrobial resistance (AMR) which may lead to antimicrobial resistant infections in clinical settings. Over 70% of antimicrobials produced globally are used in animal agriculture and although the use of antimicrobial compounds for growth promotion is being banned in multiple countries, their application is projected to increase due to increased adoption of large-scale intensive farming operations. The impact of Antimicrobial Growth Promoters (AGPs) has been especially important for the poultry industry, which has expanded more than any other protein source over the past half-century. Among poultry species, chickens (Gallus gallus) account for most of the global poultry production. Thus, there is a high incentive to identify AGP alternatives for poultry that can sustain increasing demand while reducing the risk for AMR selection and its consequences for human and animal health.

Despite their widespread use, there are multiple gaps in understanding of how AGPs impact animal performance. This is significant as knowledge of the physiological mechanisms at play is likely to uncover biological targets to induce the same benefits of AGPs without the risk of AMR. Multiple antimicrobial compounds have been used to increase weight gain and feed efficiency in farm animals. These include cyclic peptides (e.g. bacitracin), ionopohores (monensin, narasin), streptogramins (virginiamycin), orthosomycins (avilamycin), and macrolides (tylosin, spiramcycin) among others. Even though these compounds differ in their antimicrobial spectrum and antibacterial mode of action, whether they increase performance through similar or different mechanisms is not clear. Several hypotheses have been advanced to explain how AGPs may contribute to host physiology, nevertheless, compound-specific mechanisms, if any, remain poorly described. Some of the generic AGP proposed mechanisms include limiting opportunistic pathogens and subclinical infections, decreased microbial competition for host nutrients, modulating host fat digestion and utilization, inhibiting the production of toxins in the gut, regulating host's immunity and inflammation, and improving nitrogen balance. Given the involvement of the gut microbiome in most of these proposed mechanisms, engineering the gut microbiome to drive the performance and health status of animals has received considerable attention in the fight against AMR.

The avian caeca are a pair of sacs at the transition between the ileum and the large intestine. Among all organs, the cecum contains the highest density of microbial biomass in chickens, and it has been implied in processes including fermentation of undigested fiber, nitrogen re-cycling, water absorption, and overall nutritional status. Analysis of the composition of cecal microbial communities in broiler chickens has shown a predictable progression through development towards a stable microbial composition. Additionally, several studies have identified correlations between members of the cecal microbiome and bird performance under antibiotic treatment and antibiotic-free conditions, as well as similarities in the microbiome effects induced by antibiotics and probiotics. While those associations support the notion that manipulating the composition of the gut microbiome can provide a path for replacing AGPs, few studies have assessed the overlap in the taxonomic and functional consequences of distinct AGPs to guide microbiome engineering efforts.

To advance understanding of possible roles of the cecal microbiome on AGP mechanisms, the present inventors used four distinct AGPs to investigate functional changes in the cecal microbiome of broiler chickens. First, they described how AGPs causing different levels of growth promotion alter the community structures and gene abundance profiles of the cecal microbiomes of treated birds. Second, they demonstrated the contribution of core microbiome members to many of the observed functional shifts. Third, to investigate how these microorganisms may affect host physiology, they generated draft metabolic reconstructions for the core microbial genera of the cecal microbiome and model and compare phenotypic traits of the corresponding communities in silico. Finally, they used untargeted metabolomics analysis to complement the metagenomics and metabolic modeling results and further validated the functional impact of one of the AGPs on growth performance.

### SUMMARY OF THE PRESENT INVENTION

In one embodiment, the invention provides methods for determining which antimicrobial growth promoter (AGP) to administer to a domesticated organism in order to promote a healthy microbiome. The method comprises a) providing a plurality of test organisms and a control organism, b) administering a regular regimen of a different AGP to each test organism for up to about one month, c) collecting DNA from the intestinal content of the test organisms and of the control organism, d) sequencing the DNA of the intestinal content of test organisms and of the control organism to obtain a taxonomic profile, and, optionally, a functional profile, thereby defining core intestinal microbiomes of the test organisms and of the control organism, e) comparing the core intestinal microbiomes of the test organisms and of the control organism, thereby determining antimicrobial resistance (AMR) gene abundance of the test organisms, and determining which test organism has the most healthy microbiome thereby determining which AGP provides the most healthy microbiome, and f) treating the domesticated organism with the AGP which provides the most healthy microbiome, wherein the domesticated organism, test organism and control organisms are of the same species. A regular regimen of an AGP is the dose and timing administration of the AGP known to a skilled artisan, typically as directed by the manufacturer. In one embodiment, the organisms are birds, fish or mammals. Typically these methods are used in domesticated animals. Examples of mammals include pigs, cows, horses, dogs, cats and humans. Examples of birds include chickens and turkeys. Examples of AGPs include bacitracin methylene disalicylate (BMD), avilamycin, virginiamycin and narasin. In one embodiment, the intestinal content is collected from the cecum. In one embodiment, the administration of a regular regimen of a different AGPs to each test organism is for about 7 days to about 2 months, more typically from 14 days to 35 days, most typically about 28 days to about 35 days. In one embodiment, the healthy microbiome includes Lactobacillus.

### BRIEF DESCRIPTION OF THE DRAWINGS

FIG. 1A. Effects of four different AGPs on bird performance and cecal microbiome composition. Average daily gain as a function of bird age calculated for five pens of 20 birds in each of the treatment groups.

FIG. 1B. Feed conversion ratio as a function of bird age. n=5 pens per treatment.

FIG. 1C. Principal component representation of the Bray-Curtis dissimilarity among 16S microbiome profiles. Each symbol represents a sample. The numbers in parenthesis indicate the fraction of the variance explained by each of the first two principal components.

FIG. 1D. ANOSIM analysis of the similarities between samples from birds of different ages and AGP treatment groups. In the heatmap, values below the diagonal represent the ANOSIM R-score. Lower values indicate more similar communities. Values above the diagonal indicate the corresponding p-value for the null hypothesis that communities are similar.

FIG. 1E. Amplicon Sequence Variant (ASV) richness for cecal samples from birds of different ages and AGP treatment groups quantified with the Chao index.

FIG. 1F. Like FIG. 1E, but for ASV diversity quantified using the Simpson index. n=15 birds per treatment in E and F. *: Mann-Whitney U p-value<0.05.

FIG. 2. Major functional categories enriched or depleted in response to AGPs relative to controls in the cecal microbiome. Each dot represents a set of functional roles significantly enriched or depleted relative to controls (FDR<0.15). Blue lines connect AGPs to significantly enriched processes according to Gene Set Enrichment Analysis (GSEA). Red lines connect AGPs to significantly depleted processes. Labels capture the common functionality in select clusters of enriched terms. AVI: Avilamycin, VIR: Virginiamycin, NAR: Narasin, AMR: Antimicrobial resistance, AA: Aminoacid, ED: Entner Duodoroff, Mo, Molybdenum.

FIG. 3. Correlation between gene content and relative abundance contributions of core genera. Rows in the figure indicate functional terms (subsystems) significantly enriched by different treatments when considering only genes assigned to core genera of the cecum microbiome. Each dot represents a core microbial genus and its corresponding value on the x-axis shows the spearman correlation between the relative abundance of the genus and its contribution to the abundance of the corresponding functional term across all samples. The functional contribution is calculated as the mean copy number contributed by the genus across all the genes in the subsystem. Dot colors indicate the Spearman correlation P-value. BMD: Bacitracin Methylene Disalicylate, AVI: Avilamycin, VIR: Virginiamycin, NAR: Narasin.

FIG. 4A. AMR gene abundance in the cecal microbiome of birds treated with different AGPs. Total AMR gene abundance (RPKM) as a function of treatment group.

FIG. 4B. Number of distinct AMR genes detected as a function of treatment group. *Mann-Whitney p-value<0.05.

FIG. 4C. ANOSIM analysis of the Bray-Curtis dissimilarities between samples in different treatments based on their AMR profiles. In the heatmap, values below the diagonal represent the ANOSIM R-score. Lower values indicate more similar AMR profiles between treatments. Values above the diagonal indicate the corresponding p-values for the null hypothesis that AMR profiles are similar between treatments.

FIG. 4D. Abundance of AMR genes of distinct classes for samples in different treatment groups (colors). Classes are sorted according to their mean AMR gene abundance across samples. Color arrows indicate a significant increase (pointing up) or decrease (pointing down) in the abundance of AMRs in the treatments represented by the colors compared to control samples. MLS, macrolides, lincosamides, and streptrogramin A and B.

FIG. 5A. Pan-genome scale metabolic modeling of core cecum microbial genera. Predicted metabolic properties of the core cecum microbiome. The predicted ability of each core genera to use individual compounds as main carbon sources for biomass synthesis. Core genera are shown in the columns grouped by taxonomic family. Each row represents a distinct carbon-containing compound.

FIG. 5B. Like FIG. 5AA, but for the ability to use individual compounds as main nitrogen sources.

FIG. 5C. The number of carbon sources with higher or lower utilization potential in samples from each of the AGP treatments compared to controls.

FIG. 5D. Like C but for compounds used as nitrogen sources.

FIG. 5E. The total metabolic demand for core microbiome biomass synthesis across treatments. The total metabolic demand represents the likelihood that any given metabolite is essential for the growth of a random member of the core cecum microbiome. *Mann-Whitney U p-value<3×10-3. C: control, B: BMD, A: avilamycin, V: virginiamycin, N: narasin.

FIG. 6A. Cecum and Serum metabolites altered by narasin treatment. The median-normalized ion counts (scaled intensity) of 20 amino acids in the cecum of control and narasin treated birds. *Welch's t p-value<0.05, n=15 birds per treatment.

FIG. 6B. The scaled intensity of urea in the serum of control and narasin treated birds. n=10 birds per treatment.

FIG. 6C. The correlation between the abundance of dipeptides and amino acids in the cecum and bird weight at day 35 for control and narasin treated birds. Dashed red lines represent the correlation threshold for p-values<0.05.

FIG. 6D. The correlation between the abundance of amino acids in the serum and bird weight at day 35 for control and narasin treated birds. In panels C and D the x-axis values show the Pearson correlation between the logarithm of the scaled intensities and weight.

FIG. 7. Narasin treatment may alter nitrogen metabolism in the cecum aiding host performance. The inventors' results are consistent with a lower reliance of the core cecal microbiota on host protein for growth in narasin treated birds compared to controls. This is supported by several observations in the narasin treatment: 1) A higher relative abundance of urea degradation and lower relative abundance of mucin polysaccharide degradation genes. 2) A lower concentration of amino acids and dipeptides in the cecum and lower urea in the serum. 3) The positive correlation between serum amino acid levels and bird weight. And 4) A higher predicted ability to use urea and ammonia as main sources of nitrogen for biomass production.

FIG. 8A. Functional and taxonomic microbiome differences from shotgun metagenomics data. Estimated mean genome sizes per treatment group. Mean genome sizes were estimated from the abundance of genes assigned different functional roles, normalized by the abundance of universal single copy genes. The P-value is for the Mann-Whitney U test.

FIG. 8B. ANOSIM results for the comparison of functional profiles across treatment groups. Values above the diagonal represent ANOSIM P-values. Values below the diagonal represent ANOSIM R-scores.

FIG. 8C. Like B, but for the comparison of species-level taxonomic profiles obtained from the shotgun metagenomics data.

FIG. 9A. Core genera of the broiler cecum microbiome. Bacterial genera identified from metagenomics reads were sorted according to their average relative abundance across samples. The figure shows the mean fraction of classified reads accounted for the top n most abundant genera. The mean is calculated across all 74 samples from day 35 broilers.

FIG. 9B. Core genera of the broiler cecum microbiome. Bacterial genera identified from metagenomics reads were sorted according to their average relative abundance across samples. The figure shows the functional role relative abundances. The dashed red lines show that the top 25 most abundant genera account for ˜90% of classified reads and ˜75% of gene abundances.

FIG. 9C. Relative abundance of core genera as a function of treatment. P-values are for the Mann-Whitney test.

FIG. 10. Fraction of functional roles assigned to core genera shared between samples from different treatments. The figure shows the median overlap between assigned functional roles to each treatment for all pairwise treatment comparisons. Data are shown for each of 25 core genera sorted by their mean abundance across all samples. Overlap was calculated as the number of shared roles between treatments divided by the number of roles in the treatment with the lowest relative abundance for the corresponding genus. The red line indicates the median overlap (0.77).

FIG. 11. Contribution to virulence and AMR terms enriched by virginiamycin by core genera. Each plot corresponds to a virginiamycin enriched functional term. The y-axis shows the mean abundance associated with core genera (in units of mean copy numbers per genome) of genes in the corresponding function. Results for each treatment represent the mean across samples in that treatment. Colors indicate the contribution to function abundances by genes assigned to the corresponding core genera.

FIG. 12A. Urea and ammonia utilization potential and essentiality for the core cecum microbiome across treatments. The potential for urea utilization as a main nitrogen source for biomass synthesis by the core cecum microbiome.

FIG. 12B. Like FIG. 12A, but for the ability of using ammonia as a main nitrogen source.

FIG. 12C. The metabolic demand of urea as a nitrogen source for growth. Values represent the probability that urea is essential for growth for a random member of the core cecal microbiome.

FIG. 12D. Like FIG. 12C, but for the essentiality of ammonia as a nitrogen source.

FIG. 12E. The contribution of individual genera from the core cecum microbiome to the utilization potential of urea as a main nitrogen source. Values represent the relative abundance in the core of genera predicted to use urea as a nitrogen source for growth. The P-values are for the Mann-Whitney U test. C: Control. A: Avilamycin, V: Virginiamycin, N: Narasin.

FIG. 13A. Differentially abundant metabolites in the cecum of narasin treated birds. Scaled intensities of dipeptides in the cecum of control and narasin treated birds. *p-value<0.05.

FIG. 13B. Scaled intensities for the concentration of biliverdin and bilirubin in the cecum.

FIG. 13C. Scaled intensities for the concentration of riboflavin in the cecum (left) and estimated riboflavin demand by the core cecum microbiome in control and narasin treated birds (right).

FIG. 13D. Scaled intensities for the concentration of spermidine in the cecum and serum (left) and estimated spermidine demand by the core cecum microbiome in control and narasin treated birds (right). The P-values for metabolite scaled intensities are for Welch's T test, P-values for estimated metabolite demands are for the Mann-Whitney U test.

FIG. 14. Abundance of bile salt hydrolase homologues across treatments. Values are expressed as reads per kilobase per million reads (RPKM) corresponding to reads mapped to regions in assembled contigs with homology to bile salt hydrolases. BLASTX 78 was used to identify regions in the assembled contigs for each sample with >50% sequence identity over 70% of the length of UniProt 79 proteins annotated as bile salt hydrolases or chloroylglycine hydrolases. The indicated P-value is for the Mann-Whitney U test.

FIG. 15A. Comparison of metabolic demand predictions for different parameters in the simulation of in silico media. Results are shown for the average essentiality for growth of metabolites in the core microbiome (Top row). The columns show results for different parameter combinations used for simulating random in silico media to test essentiality. Data are shown for 90% and 80% probabilities of metabolites with transporters being included in the in silico media (columns 1 and 2), as well as 90, 80% and 70% probabilities for metabolites with transporters combined with 10%, 30% or 50% probabilities for metabolites without transporters (columns 3, 4 and 5). P-values are for the Mann-Whitney U test. C: control, B: BMD, A: avilamycin, V: virginiamycin, N: narasin

FIG. 15B. Comparison of metabolic demand predictions for different parameters in the simulation of in silico media. Results are shown for the average essentiality of urea for growth (Bottom row).

### DETAILED DESCRIPTION OF THE PRESENT INVENTION

Antimicrobial growth promoters (AGPs) have played a decisive role in animal agriculture for over half a century. Despite concerns about antimicrobial resistance and mounting demand for antibiotic alternatives, a thorough understanding of how these compounds drive animal performance and whether those mechanisms can be exploited via alternative routes is missing.

In one embodiment, the present invention provides methods of selecting an antimicrobial growth promoter (AGP) treatment for an organism (e.g., animal) in order to promote a healthy microbiome. Animals which can benefit from the methods of the present invention include birds, humans, or non-human mammals. Examples of animals include pets (e.g., dogs, cats, etc.), livestock (e.g., cows, cattle, pigs, etc.), birds and laboratory animals (e.g., rodents, primates, etc.). Specific examples of birds include poultry such as chickens or turkeys. Examples of chicken include broiler chickens or egg-laying or egg-producing chickens.

In one embodiment, the method comprises providing at least one test animal and a control animal. The test animal(s) and control animal are preferably of the same species. A regular regimen of at least two different AGPs are administered to each test animal for an assessment period of about 1-2 weeks to about two months, more typically, about 35 days. A regular regimen of an AGP is the dose and timing administration of the AGP known to a skilled artisan, typically as directed by the manufacturer.

Once the assessment period has ended, DNA is collected from the intestinal content of the test animal(s) and of the control animal. Preferably, the intestinal content is collected from the cecum.

The DNA of the intestinal content of at least one test animal and of the control animal is then sequenced. From the DNA, a taxonomic profile, and, optionally, a functional profile, is then produced. A taxonomic profile provides the taxonomic composition of the analyzed sample, and the relative abundances of organisms. A functional profile describes gene and protein functions and interactions. From such profile(s), the core intestinal microbiomes of the test animal(s) and of the control animal are obtained.

The core intestinal microbiomes of the test animals(s) and of the control animal are then compared to determine the antimicrobial resistance (AMR) gene abundance of the test animal(s), and determining which test animal has a healthy (e.g., the most healthy) microbiome. In such manner, it is determined which AGP provides a healthy microbiome. The AGP which provides a healthy microbiome is then selected. In some embodiments, the healthy microbiome includes Lactobacillus.

Examples of typical AGPs include bacitracin methylene disalicylate (BMD), avilamycin, virginiamycin, narasin, lactam antibiotics (e.g., penicillins, lincosamides and macrolides (e.g., erythromycin, tetracyclines, kitasamycin, oleandomycin and tylosin)), flavophospholipol, pleuromutilins, quinoxalines (e.g., quinoxaline olaquindox), arsenical compounds, ionophores (e.g., monensin and lasalocid), salinomycin and avoparcin.

An AGP which provides a healthy microbiome is a microbiome which is able to synthesize biomass from more diverse carbon and nitrogen sources, and/or has fewer metabolite requirements for microbial growth. In some embodiment, an AGP which provides a healthy microbiome is associated with an increase in at least one of: metabolism of aromatic amino acids, propanediol utilization, molybdenum-containing cofactor synthesis, CO₂ fixation via the Wood-Ljungdahl pathway, protein synthesis, biotin synthesis, methionine metabolism, glycolysis, sporulation, soprenoid synthesis, betaine biosynthesis, pyridoxine biosynthesis, stress response, bacterial secretion systems, antimicrobial resistance, biosynthesis of Gram-negative cell wall components, tryptophan synthesis, urea degradation, heme/tetrapyrrole biosynthesis, bilirubin, biliverdin and xylose utilization. In some embodiments, an AGP which provides a healthy microbiome is associated with at least one of: decreased metabolites implicated in amino acid metabolism, decreased long-chain fatty acids, increased levels of metabolites from primary and secondary bile acid metabolism, increased levels of metabolites related to hemoglobin and porphyrin metabolism, increased levels of metabolites related to fructose and galactose metabolism, increased levels of tocopherol, inositol, riboflavin, nicotinate and/or nicotinamide metabolism. In some embodiments, an AGP which provides a healthy microbiome is associated with decreased levels of microbial genes associated with degradation of matrix polysaccharides and BSH genes, and/or higher levels of riboflavin synthase genes.

In another aspect of the present invention, once it is determined which AGP provides a healthy (e.g., the most healthy) microbiome, such AGP is administered to animal(s) to promote a healthy microbiome and to treat the animal(s).

In one embodiment, the present invention provides the functional footprint of microbial communities in the cecum of chickens fed four distinct types of AGPs and identifies metabolic traits that positively impact animal health and performance. The results show that different AGPs change the composition and functional capacity of the cecum microbiome in unique ways compared to controls, with relatively few taxa, metabolic or antimicrobial resistance (AMR) genes similarly altered across treatment groups. It is shown that changes in the gene content of the cecal microbiota across AGP treatments are often driven by differences in the abundance of core microbiome members. Constraints-based modeling of 25 core bacterial genera in the cecum show that the AGPs producing the largest performance effects were associated with cecal microbiomes with higher biosynthetic capabilities and fewer metabolite demands for microbial growth. Analyses show that altered nitrogen utilization by the cecum microbiome as a mechanism of narasin, the most effective AGP in the study. Untargeted metabolomics of the cecum and serum of narasin treated birds were consistent with model predictions, so that members of the core cecum microbiome can be targeted for enhanced performance through their contribution to host-microbiota metabolic crosstalk.

### AGPs Significantly Improved Weight Gain and Feed Conversion Efficiency of Broilers.

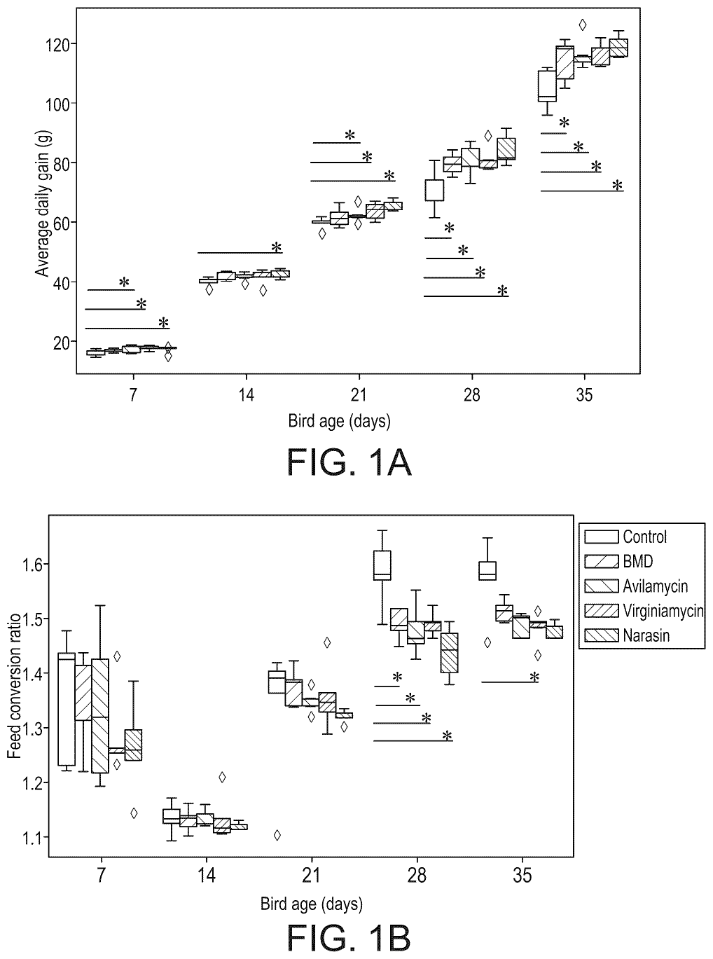

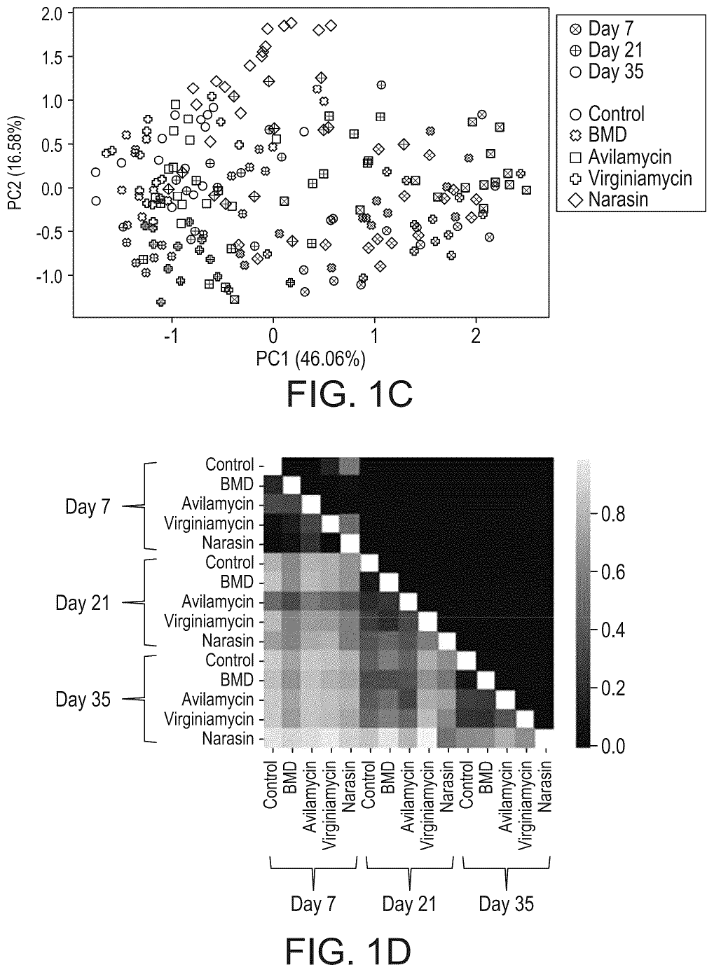

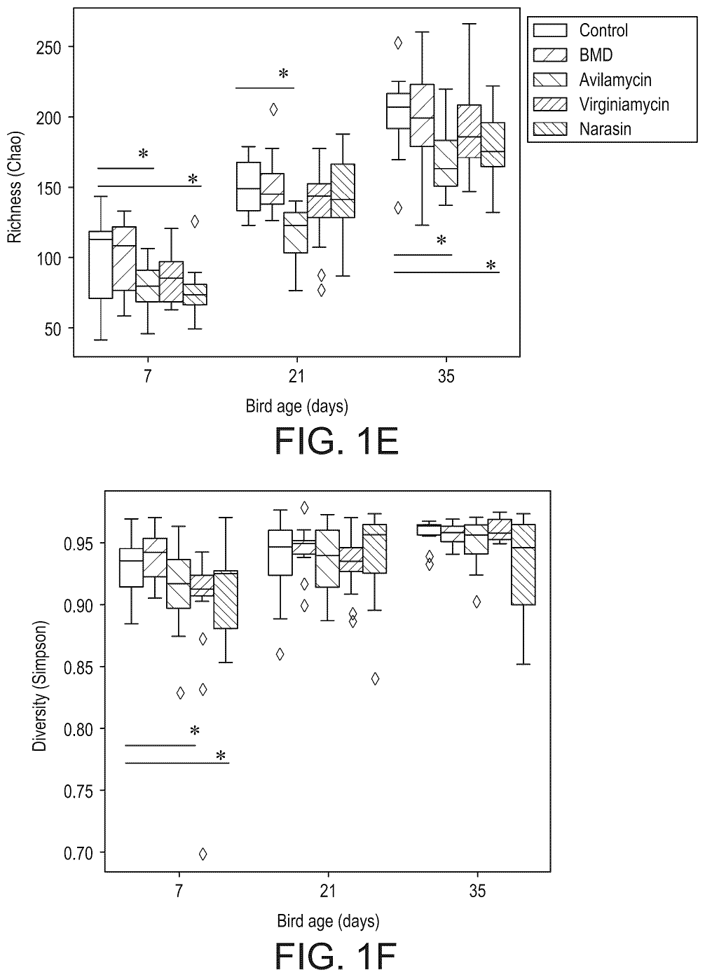

The inventors carried out a clinical study including 500 broilers randomly assigned to either a control group or to one of four in-feed AGP treatment groups (Bacitracin methylene disalicylate (BMD), avilamycin, virginiamycin or narasin). Compared to untreated controls, broilers receiving AGPs in the diet showed increased daily weight gain and improved feed conversion efficiency (FIG. 1). These trends were present throughout the length of the study (35 days) but became visibly more marked during the finisher period (28 to 35 days). On the final week of the study, the average daily gain (ADG) was 9 to 14% higher in AGP treated animals compared to controls and the feed conversion ratio (FCR) was lower in AGP treated groups by 3.8 to 6.2%. Among AGPs, narasin had the highest impact on performance, followed by virginiamycin, avilamycin, and BMD.

### Different AGPs Lead to Distinct Cecal Microbial Community Structures.

After confirming the AGPs effects on performance, the inventors investigated how the cecal microbiome might have played a role in the observed phenotypes. For this, the cecal contents from ˜15 birds per treatment group (three from each pen) were used for 16S rRNA amplicon microbiome profiling at days 7, 21 and 35 of the study (data was obtained for 223 samples). Across samples a total 2,937 amplicon sequence variants (ASVs) were identified, with a mean of 137 ASVs per sample. The inventors observed a strong effect of bird age on microbiome composition (FIG. 1C), with samples collected on different days spanning the first two principal components of the dissimilarity matrix between samples (variance explained: ˜ 62%). Independent of age, the inventors also observed significant microbiome structure differences between samples from different treatment groups. An analysis of similarities (ANOSIM) based on the Bray-Curtis dissimilarities among 16S profiles showed that, except for a couple of treatment pairs at the earliest timepoint, all pairwise comparisons between treatments resulted in significantly different microbiome compositions at every timepoint (FIG. 1D). Interestingly, on days 21 and 35 there was a higher similarity (lower ANOSIM R scores) among control samples and samples from the AGP treatments producing the smallest effects on performance (BMD and Avilamycin). Thus, larger performance gains due AGP application were accompanied by bigger changes in cecal microbiome composition. Looking at the diversity within samples (a diversity), the effect of bird age on cecal microbial community structure was reflected by an increase in both richness and evenness as birds developed (FIGS. 1E, F). However, the application of AGPs tended to reduce microbial richness at all three timepoints, as well as evenness (Simpson diversity) in the case of day-7 samples.

Altogether, the ANOSIM and a diversity results indicate that each AGP led to the development of a distinct cecal microbial community structure. To investigate how these mature communities may have contributed to the observed performance gains, the inventors used shotgun metagenomics to analyze the gene content of microbes in the cecum of day 35 broilers across treatment groups.

### Partially Overlapping Functional Pathways are Enriched by Different AGPs.

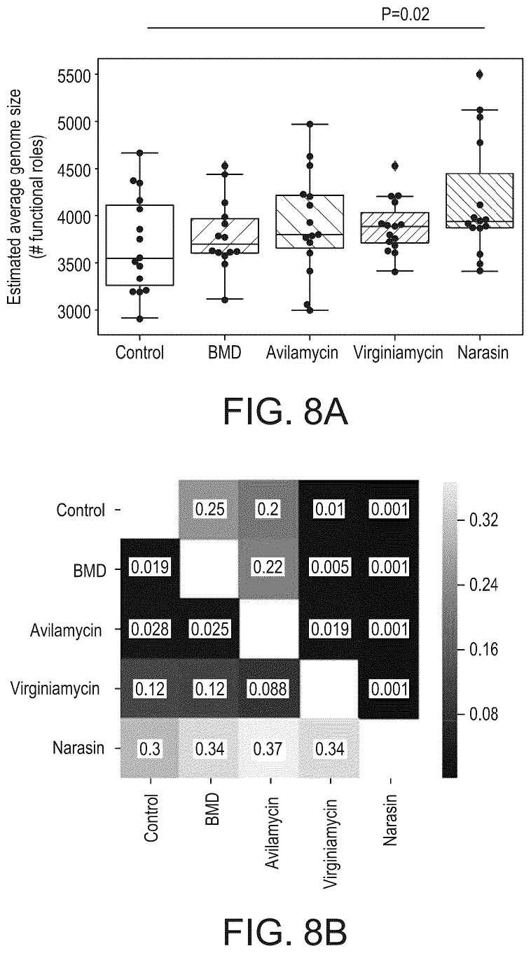

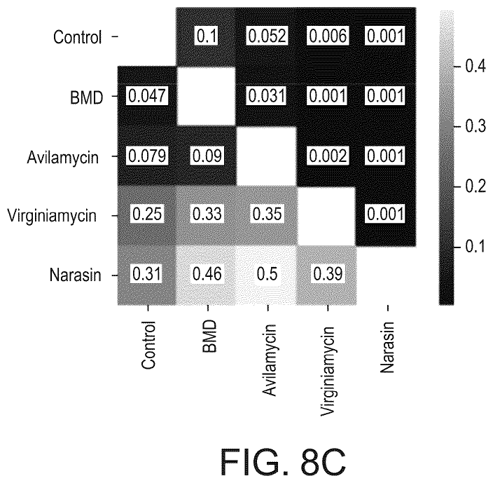

Sequencing reads from 74 day-35 samples were assembled into contigs and annotated for protein coding genes. The predicted genes were functionally annotated using RAST functional roles, and role abundances per sample were expressed in terms of mean copy numbers per genome (see methods). Interestingly, the normalized data revealed a trend of increasing gene content per genome in the AGP treatments compared to controls, which was significant in the case of narasin (Mann-Whitney U p-value=0.02, FIG. 8A). Additionally, based on the Bray-Curtis dissimilarities between functional profiles the inventors observed a trend of increasing functional distance with increasing AGP effects on performance (FIG. 8B). Specifically, the ANOSIM R scores between profiles in the control, BMD and avilamycin groups were smaller than the corresponding distances to the virginiamycin or narasin groups. Furthermore, in agreement with the 16S results, taxonomic profile distances calculated from the shotgun metagenomics data showed mostly significant differences between treatments (ANOSIM p-values<0.05) and larger differences (ANOSIM R scores) for the AGPs producing the highest effects on growth (FIG. 8C).

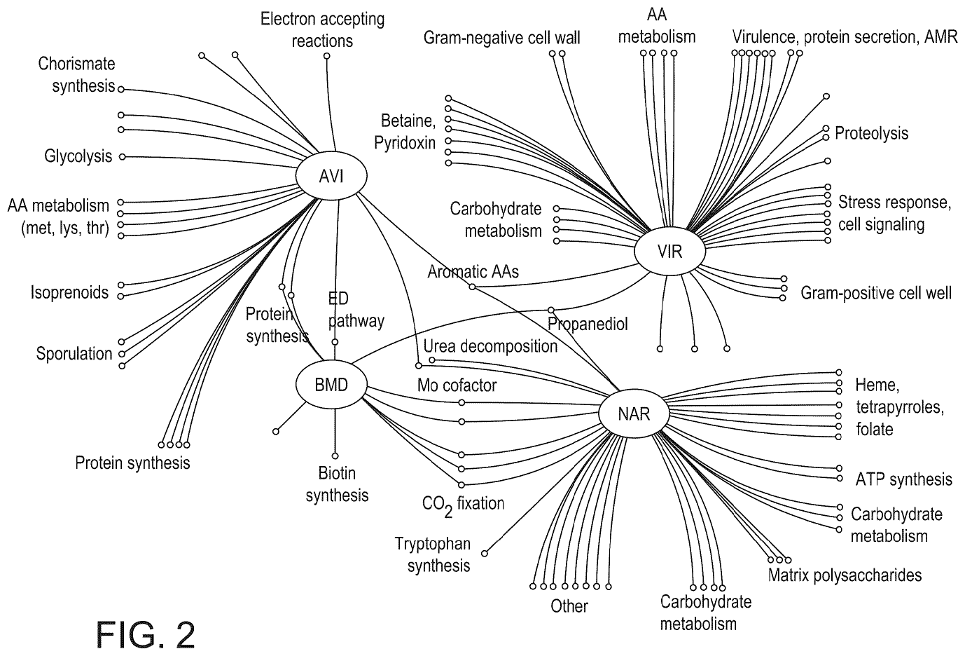

To investigate in detail how AGP treatment altered the functional makeup of the cecal microbiome, the inventors carried out gene set enrichment analysis (GSEA) to identify subsystems or pathways enriched by each AGP compared to control birds. FIG. 2 depicts the major functional processes whose abundance was enriched (blue) or depleted (red) in the cecal microbiome of AGP treated birds. Notably, each treatment pointed to a distinct collection of enriched or depleted gene functions, with about 8-fold more functional categories changed by a single AGP than by two or more AGP treatments. Consistent with the ANOSIM results, there were more functional terms enriched or depleted in the virginiamycin and narasin treatments, than in birds treated with BMD or avilamycin. Among the processes independently enriched by more than one AGP were the metabolism of aromatic amino acids (all AGPs except BMD), propanediol utilization, molybdenum-containing cofactor synthesis and CO₂ fixation via the Wood-Ljungdahl pathway (BMD and narasin), and protein synthesis and the Entner-Doudoroff pathway (BMD and avilamycin). Processes enriched by individual AGPs included biotin synthesis (BMD); methionine metabolism, glycolysis, sporulation, and isoprenoid synthesis (avilamycin); betaine and pyridoxine biosynthesis, stress response, bacterial secretion systems, antimicrobial resistance (AMR), and biosynthesis of Gram-negative cell wall components (virginiamycin). Tryptophan synthesis, urea degradation, heme and tetrapyrrole biosynthesis, and xylose utilization among others, were enriched by narasin. Narasin also led to the depletion of functional roles associated with the utilization of mucin polysaccharides as well as cell matrix components including sialic acid, N-acetylneuraminate and hyaluronic acid.

In addition to gene functions enriched between AGP treatments and controls, the inventors looked for functions enriched according to the correlation across samples between normalized gene functional role abundances and the weight at day 35 of corresponding birds within each treatment. In general, the processes enriched according to the magnitude of the correlation with weight were similar to those enriched in the comparisons to controls. Specifically, ˜25% of gene sets significantly enriched by at least one AGP relative to controls, were also significantly enriched (p<0.05) by a positive correlation with weight in at least one of the five treatment groups. These gene sets comprise roles associated with protein synthesis, the Wood-Ljungdahl pathway, tryptophan synthesis, sporulation, glycolysis, tetrapyrrole synthesis, and urea degradation. Similarly, 50% of depleted terms in the comparison to controls showed a negative correlation with weight in at least one treatment group. These include the utilization of sialic acid, hyaluronic acid and N-acetylneuraminate. Interestingly, while several terms were enriched by both methods for samples from the same treatment (e.g. protein synthesis in BMD or glycolysis and sporulation in avilamycin), several of the processes enriched by narasin also showed up as positively associated with weight in other treatments. Namely, tetrapyrroles and tryptophan synthesis (avilamycin), and urea degradation (control). These results suggest that these processes could potentially play a role in broiler performance regardless of the treatment that induces them.

### The Core Microbiome Accounts for a Large Fraction of AGP-Related Functional Shifts.

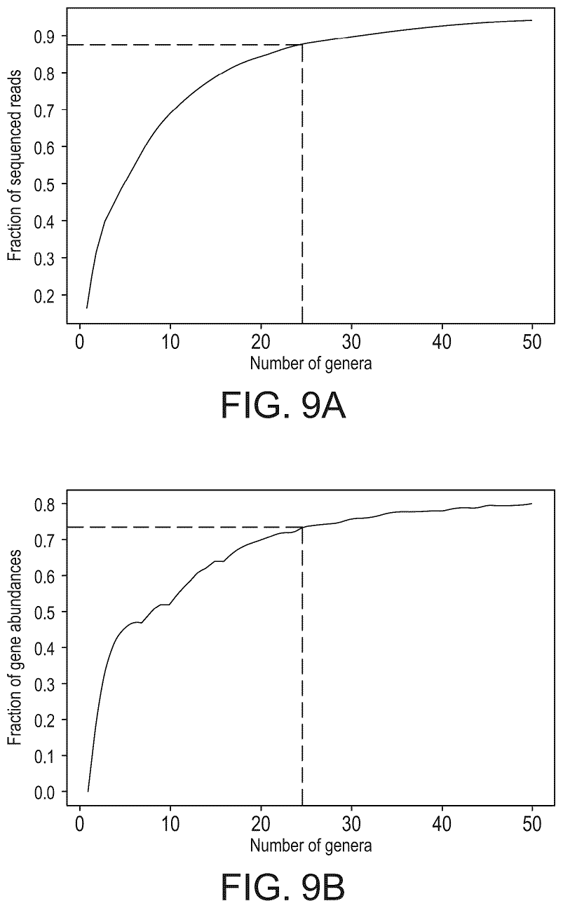

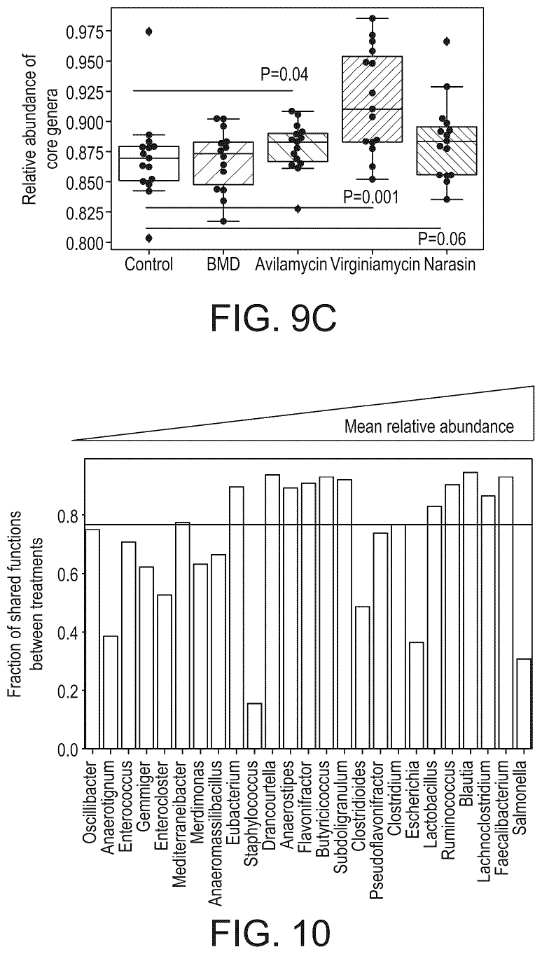

Changes in the prevalence of genes associated with a particular function in response to AGP treatment can be due to differences in either the gene content or abundance of multiple, relatively rare microorganisms or of the more prevalent and abundant members of the cecal microbiome. To check whether one of these cases dominates the functional changes observed for each AGP, the inventors repeated the above GSEA analysis using only gene abundances calculated for core members of the microbiome. Core microbiome members were defined as the top 25 bacterial genera according to their mean relative abundance across all day-35 samples (see methods). These core genera account, on average, for ˜90% of the microbial relative abundance (FIG. 9A) estimated from the metagenomics reads and represent close to 75% of functional role relative abundances (FIG. (2B). In the case of BMD and avilamycin, only 18% and 28% of GSEA enriched functions were still significant (p-value<0.05) using only gene abundances from core genera. However, this number increased to 52% and 78% in the case of virginiamycin and narasin, respectively. Thus, a substantial proportion of functional changes (61 out of 117 function enrichments), especially for the most effective AGPs, can be attributed to changes in the abundance or gene content of core microbiome members. The inventors noted that the core microbiome accounts for a slightly higher fraction of relative abundances (˜1-5%) in samples from the avilamycin, virginiamycin and narasin treatments compared to BMD and controls (FIG. 9C).

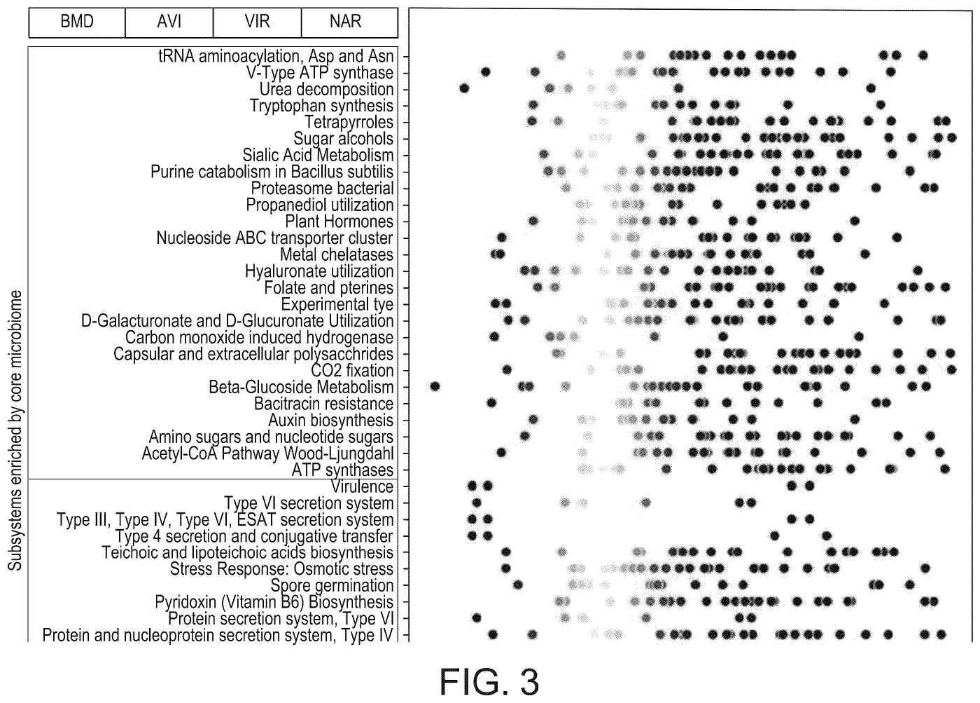

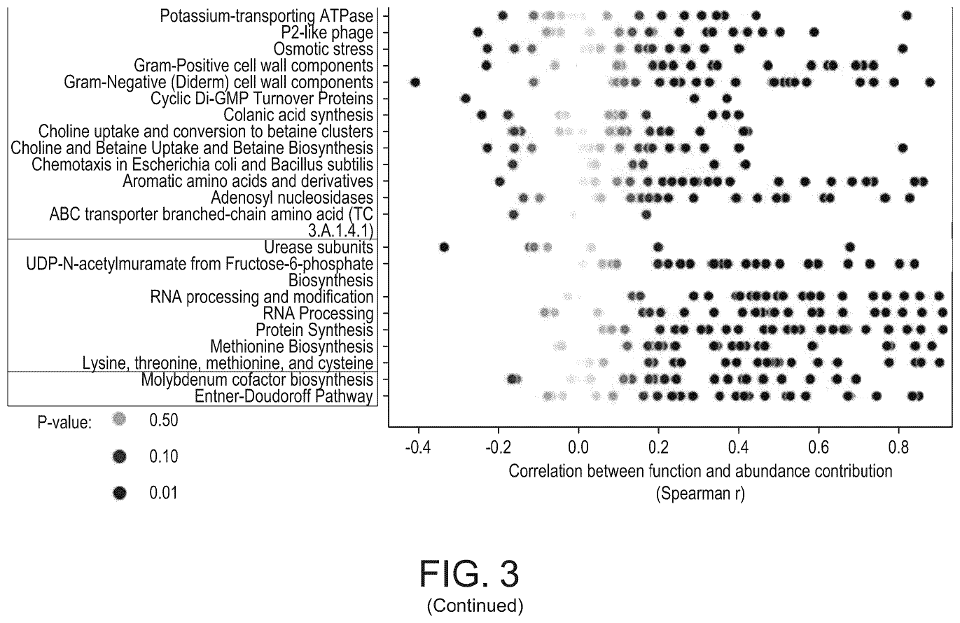

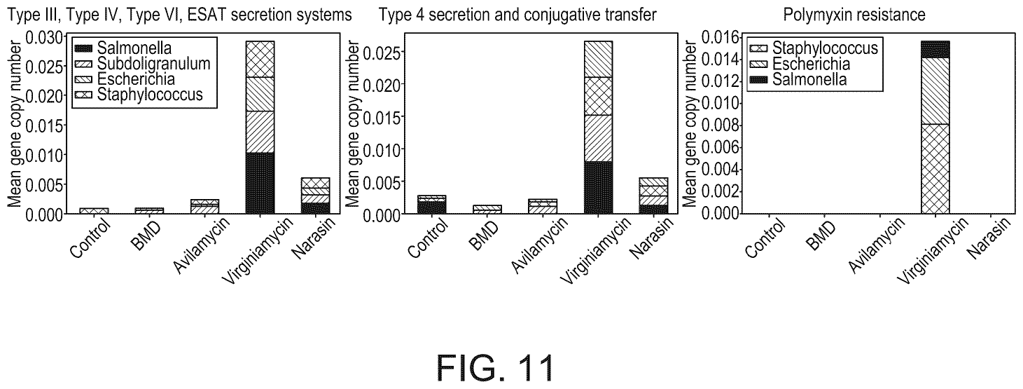

Next, the inventors asked whether functional changes across treatments were mostly driven by differences in the gene content or abundance of the core microbial genera. For each of the 59 unique functional terms also enriched by the core microbiome the inventors calculated the correlation across all samples between the relative abundance of core genera and their contribution to mean gene copy numbers of the corresponding functions. A positive correlation would suggest that functional differences between samples reflect abundance differences of microorganisms with a fixed gene content. A weak correlation, on the other hand, would indicate that functional differences are due to distinct genes being carried by the same genera across samples from different treatments. As shown in FIG. 3, most correlations were found to be positive and significant, suggesting that the genus-level gene content is likely shared across treatments and that shifts in the abundance of the corresponding functions are due to altered abundances of core genera in the cecum microbiome. As an additional test, the inventors looked at the overlap between functional roles assigned to core genera in samples from different treatments. The inventors found (FIG. 10) that about 80% of the functions assigned to a particular genus in samples from one treatment were also assigned to that genus in other treatments. For each genus, the overlap was calculated relative to the roles assigned in the treatment with the lowest relative abundance for the genus. This was done to control for gene content differences caused by changes in sequencing coverage. While this result also suggest that the genus level gene content of core genera was likely similar across all samples, the inventors observed a few outliers. Specifically, the genera Salmonella, Escherichia, Clostridioides and Staphylococcus showed a much lower gene content overlap, suggesting that AGPs could have enriched for different species and strains within these genera. Salmonella and Escherichia were found at much higher abundances in the virginiamycin treatment (˜50% relative abundance), and they are largely responsible, along with Staphylococcus, for the virulence, protein secretion systems and AMR functional roles enriched by this AGP (FIG. 11). As observed in FIG. 3, the correlations associated with virginiamycin enriched terms are lower compared to other treatments, indicating that specific strains might have contributed to the differential abundance of these functions with virginiamycin.

Altogether the results indicate that changes in abundance of core microbiome genera induced by AGP treatment drove a large fraction of the gene function abundance differences observed in the cecum microbiome.

Different AMR Gene Abundance Profiles Upon Treatment with Distinct AGPs.

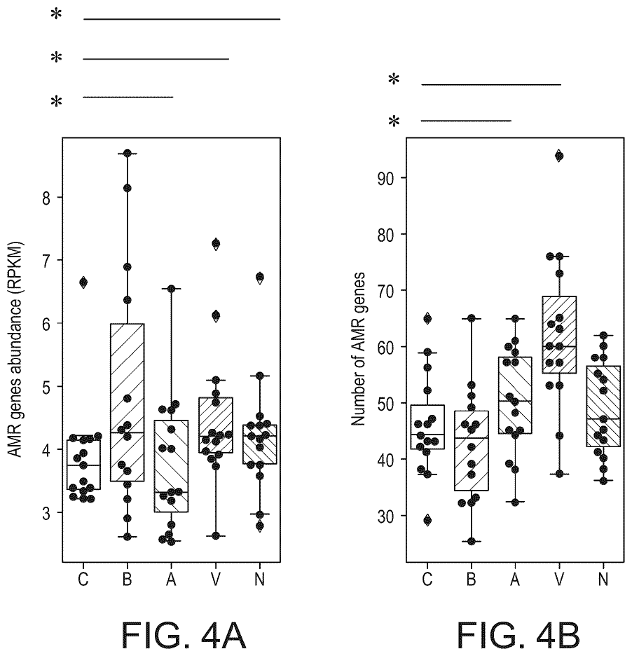

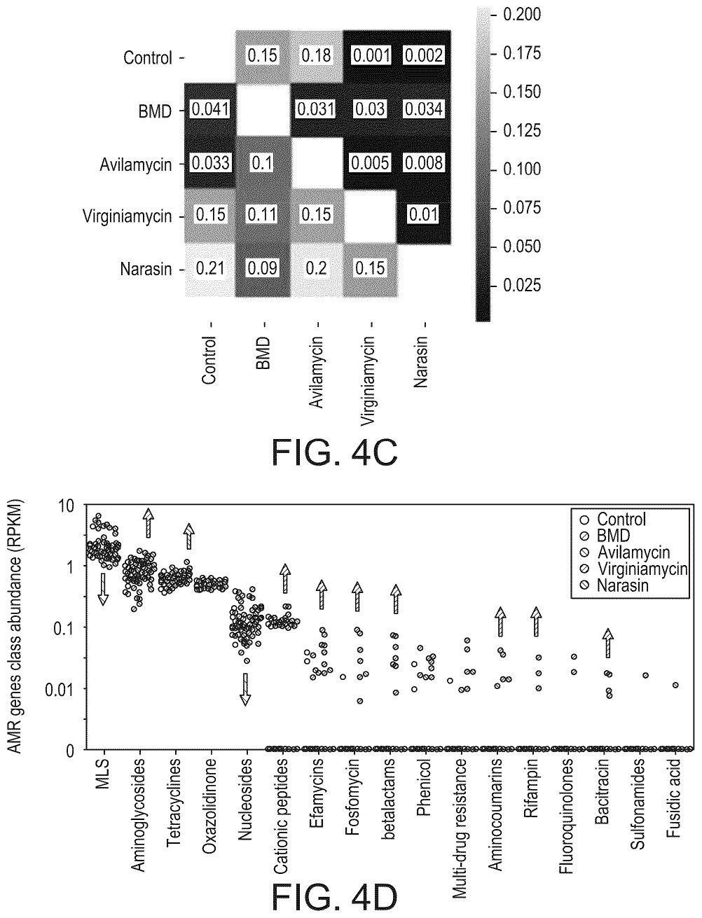

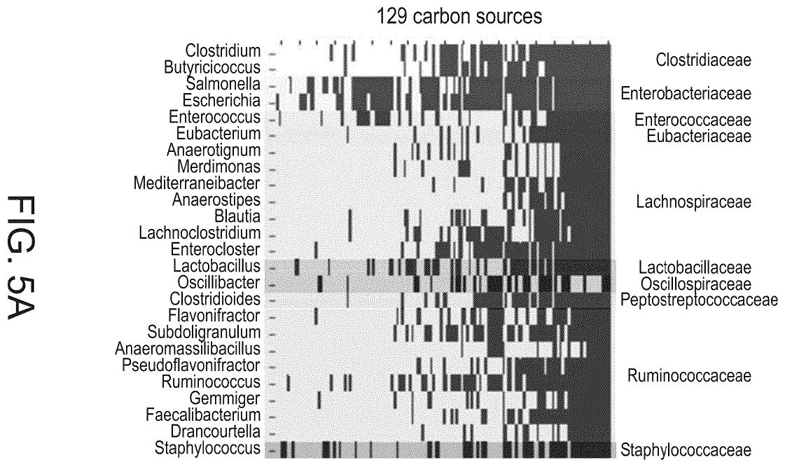

To further investigate the impact of AGPs on the prevalence of AMR genes in the cecum microbiome the inventors used the AMR . . . (Lakin et al., Nucleic Acids Res 45, D574-D580 (2017)) pipeline to estimate AMR gene abundances across samples. The analysis revealed a significant increase in total AMR gene abundance in the BMD, virginiamycin and narasin treatments compared to control (FIG. 4A). Additionally, the total number of different AMR genes detected was higher in the avilamycin and virginiamycin groups (FIG. 4B). Interestingly, looking at the similarity between treatments based on their AMR gene profiles, the inventors observed similar patterns to those found when comparing taxonomic or functional profiles (FIG. 4C). Namely, the AMR gene profiles were most similar between control and AGPs producing the smaller effects on performance, and least similar for virginiamycin and narasin compared to the remaining treatments. Compared to controls, the virginiamycin cecal microbiome enriched (Mann-Whitney U p-value<0.05) for genes involved in resistance to multiple drugs (aminoglycosides, bacitracin, cationic antimicrobial peptides, elfamycins, fosfomycins, rifampin, betalactams and aminocumarins). Narasin, on the other hand, enriched for resistance to tetracyclines. Interestingly, avilamycin reduced the abundance of genes associated with resistance to nucleosides and macrolides (FIG. 4D). In conclusion, the effect of AGPs on AMR profiles was minor for BMD and avilamycin, and, similar to taxonomic and functional profiles, treatment with virginiamycin and narasin led to significantly different AMR gene abundance profiles.

The inventors reasoned that the metabolism and growth of the most abundant members of the microbiome is likely to determine the bulk of metabolic demands and byproducts of the cecum microbiome. Specifically, metabolic fluxes driven by low abundance microbes (non-core, or <0.5% relative abundance) would have to be comparatively large to counteract the effects of core microbiome members representing 90% of microbial abundances. Given that the genome composition of these core microbes seems largely conserved across samples, the inventors built individual metabolic reconstructions for each of the 25 core genera based on the gene functions assigned to those genera across samples (see methods). The inventors then used constraints-based modeling (Orth et al., Nat Biotechnol 28, 245-248 (2010)) to investigate possible functional impacts of their abundance changes. As expected, more genes were assigned through the metagenome annotations to the more abundant core genera (Spearman r: 0.49, p-value: 0.01). This is likely the effect of sequencing depth, as the inventors found no significant correlation between the gene content of representative sequenced genomes from core genera and their mean abundance in the cecum (r: 0.12, p-value: 0.55). Thus, the inventors supplemented the metagenome annotations based on the metabolic gene content of fully sequenced reference genomes to obtain draft metabolic reconstructions of the expected size for each of the core genera. The resulting genus-level reconstructions ranged from 927 reactions for Anaerotignum to 1634 reactions for Escherichia. The inventors noticed a positive correlation (r: 0.64, p-value: 6E-4) between the number of species with sequenced genomes in each core genera and the size of their corresponding reconstructions. This could possibly be a result of gut microbes with smaller genomes being less readily culturable and less often sequenced.

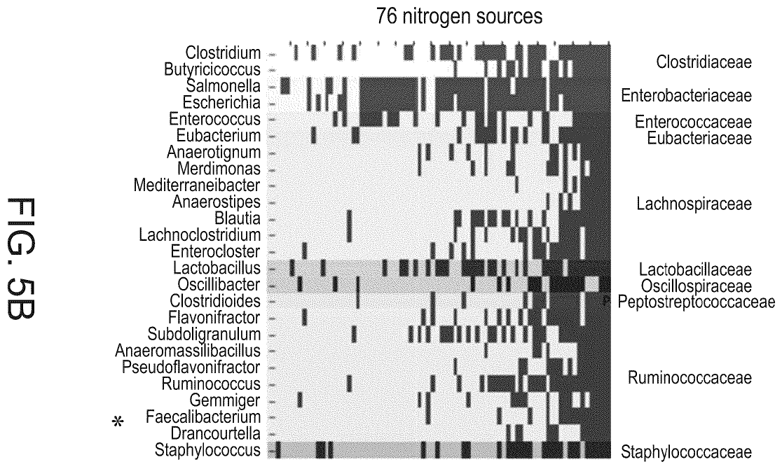

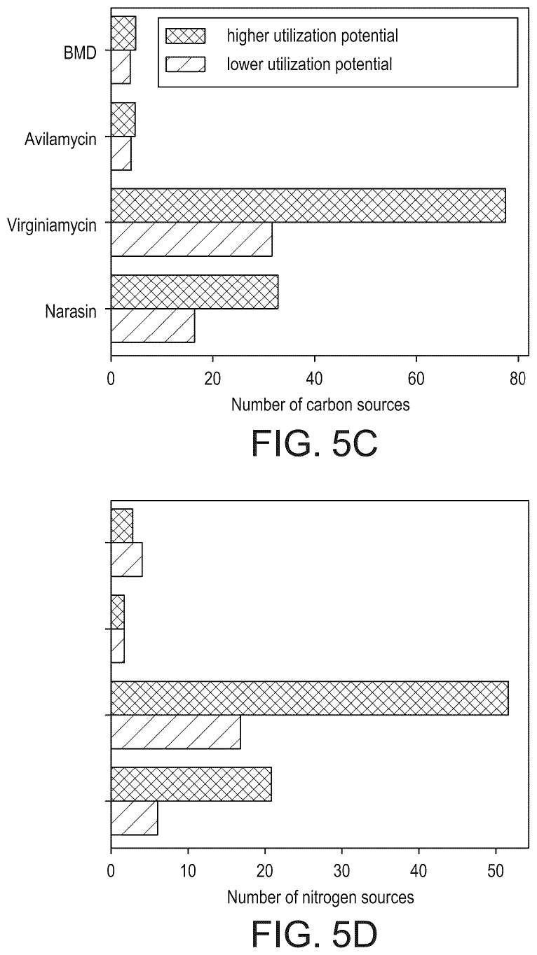

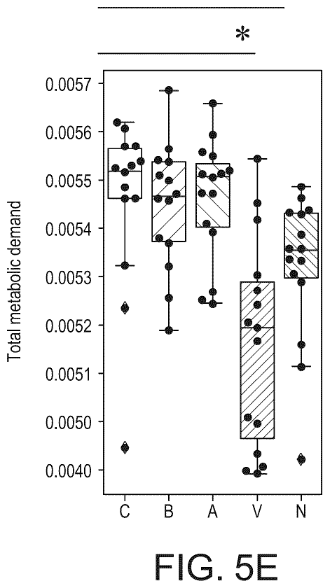

To characterize the potential impact of the metabolism of core microbes on metabolite pools in the cecum, the inventors first studied their ability to use different carbon and nitrogen sources. The inventors simulated the ability of each model to utilize one of 129 and 76 compounds as the main carbon or nitrogen source for growth, respectively (see methods). As shown in FIGS. 5A and 5B, the predictions varied substantially across genera, suggesting that members from different genera in the cecum have unique metabolic capabilities. As expected from their larger gene sets, the pan-genome reconstructions for Escherichia, Salmonella, Clostridium and Ruminococcus, predicted a higher number of carbon and nitrogen compounds that could support growth. Given the observed differences in metabolic versatility between genera, the inventors asked how differences in the relative abundances of these microbes across treatments may impact the metabolic potential of the core cecum microbiome. For each sample, the utilization potential of a particular carbon or nitrogen source was calculated as the relative abundance in the core microbiome of the genera able to use it for biomass synthesis. Core relative abundances were calculated by dividing the abundance of each core genus by the total abundance of core genera in each sample; this was done to account for the slightly different representation of core genera across treatments. In general, the inventors found more compounds with significantly higher carbon and nitrogen utilization potentials in AGP treated birds compared to controls than compounds with lower utilization potentials (FIG. 5C, D). The inventors also found more compounds with significantly different utilization potentials compared to controls in the virginiamycin and narasin treatments than in the BMD and avilamycin treatments. These results support the idea that metabolic versatility of the core cecum microbiome was increased following virginiamycin and narasin treatment. Also, the larger differences in predicted phenotypic properties of core genera align with differences in both taxonomic composition and functional gene content in the AGPs with the largest performance effects.

To further explore the metabolic consequences of core microbiome composition and functional potential, the inventors predicted essential nutrients for growth for each of the core genera (see methods). Each genus was characterized by a mean metabolite essentiality value representing the probability that a metabolite is essential for growth across random nutritional environments. For each sample the inventors then calculated a total metabolic demand of the core microbiome by summing the mean metabolite essentiality of core genera weighted by their relative abundance (FIG. 5E). The results showed a significantly lower overall metabolic demand of the core microbiome in the narasin treatment; that is, a lower likelihood that any single nutrient is required for the growth of a random member of the core microbiome. These results agree with the above prediction of a higher potential to use distinct nutrients as carbon or nitrogen sources.

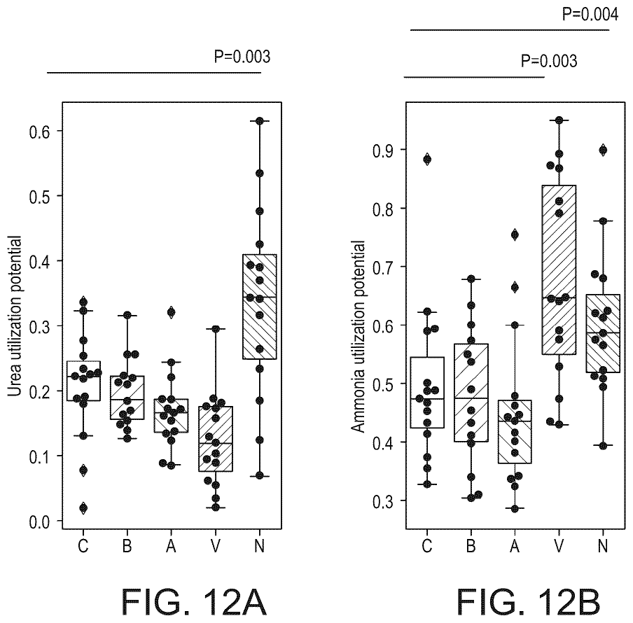

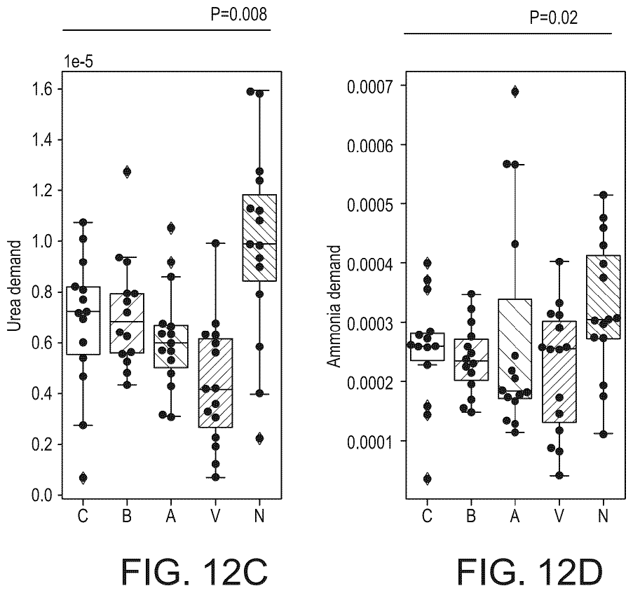

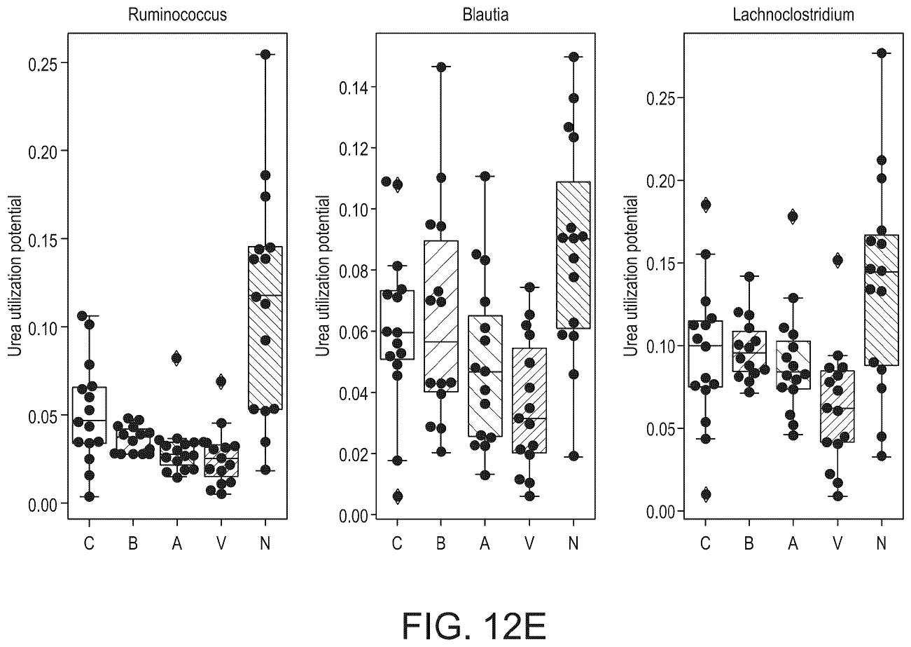

Interestingly, the inventors found that the utilization potential of urea as a nitrogen source for biomass synthesis was higher in the narasin treatment (FIG. 12A). A similar result was observed for ammonia with both virginiamycin and narasin (FIG. 12B). Not only was there a higher predicted capacity of the core cecum microbiome to use urea with narasin, but urea was also more often essential for growth in this treatment (FIG. 12C). Moreover, the genus-level metabolic reconstructions allowed the inventors to look at the contributions of each of the core genera to the above sample-level predictions. The inventors' analyses suggest that the higher urea utilization potential in the narasin treatment is partly driven by higher abundances of Ruminoccocus, Blautia and Lachnoclostridium (FIG. 12D).

### Untargeted Metabolomics Supports a Role of the Narasin Microbiome on Nitrogen Balance.

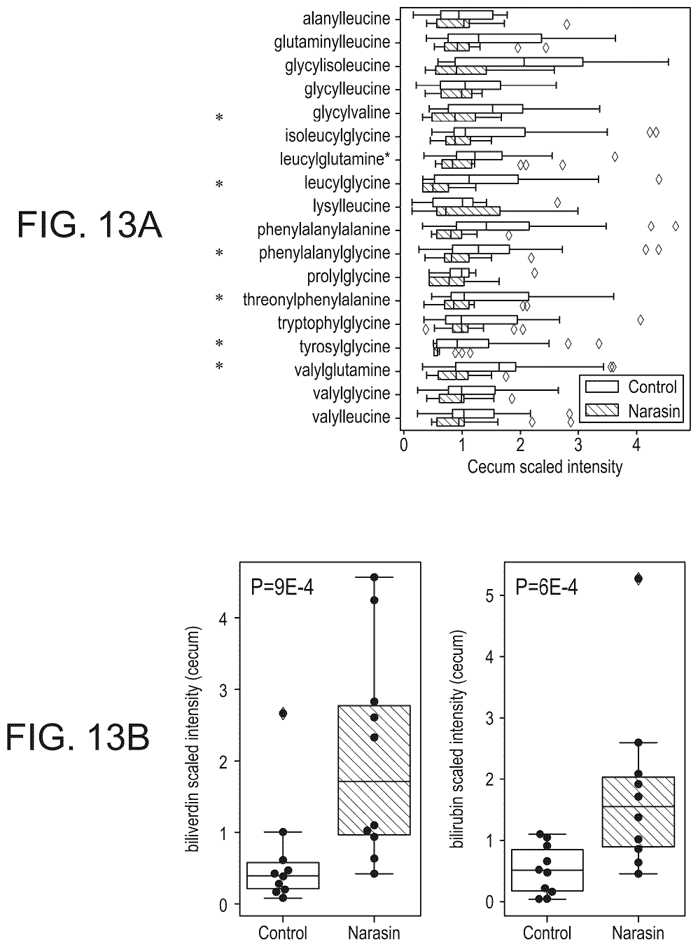

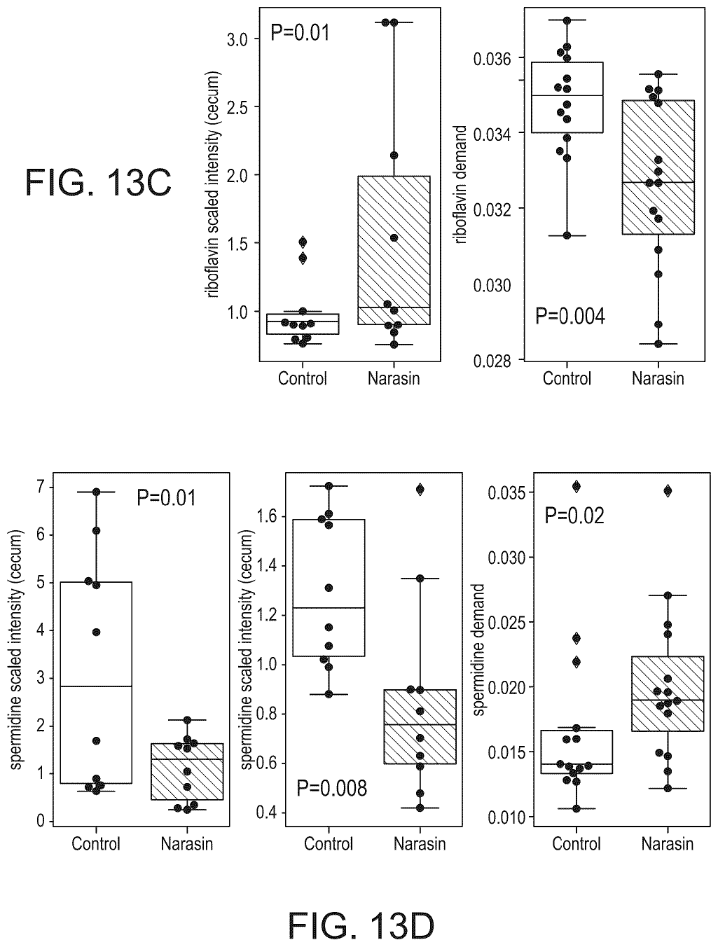

To obtain further evidence of the metabolic consequences of cecal microbiome differences due AGP treatment, the inventors performed untargeted metabolomics on the cecum and serum of control and narasin-treated birds. Altogether, 712 and 723 named metabolites were identified across 30 cecal and 20 serum samples, respectively. Of these, 49 were found at higher abundance and 78 at lower abundance in the cecum of narasin treated birds compared to controls (Welch's t-test, Benjamini-Hochberg corrected p-value<0.05). In the serum, 4 metabolites were found at higher abundance and 93 at lower abundance in narasin treated birds. To broadly characterize these metabolites, the inventors ran GSEA ranking all detected metabolites according to their standardized difference between treatments (see methods). In the cecum, dipeptides (FIG. 13A) and metabolites implicated in amino acid metabolism were significantly depleted by the narasin treatment, along with several long-chain fatty acids (FDR<0.15). Metabolites related to hemoglobin and porphyrin metabolism, fructose and galactose metabolism, tocopherol, inositol, riboflavin and nicotinate and nicotinamide metabolism were enriched. In the serum, phospholipids, fatty acids, and metabolites associated with secondary bile acid metabolism, heme degradation and vitamin A metabolism were enriched, while hexosylceramides and metabolites in the urea cycle, arginine and proline metabolism were depleted.

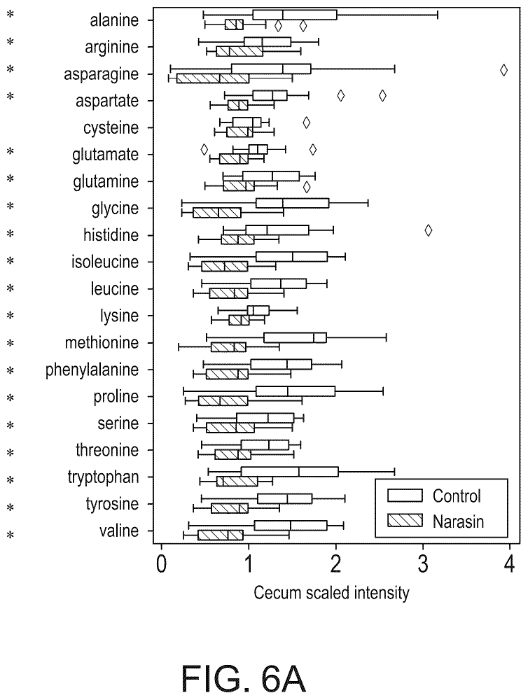

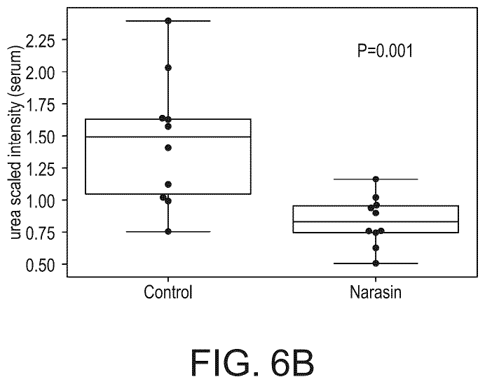

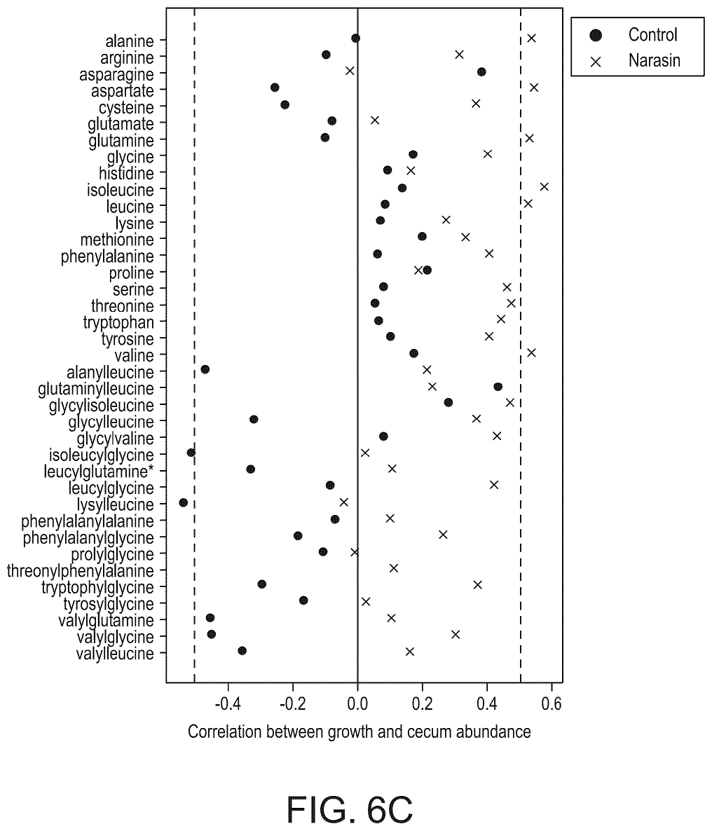

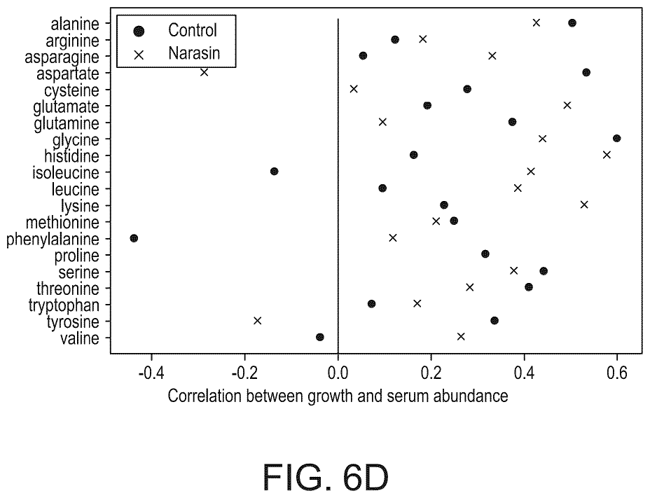

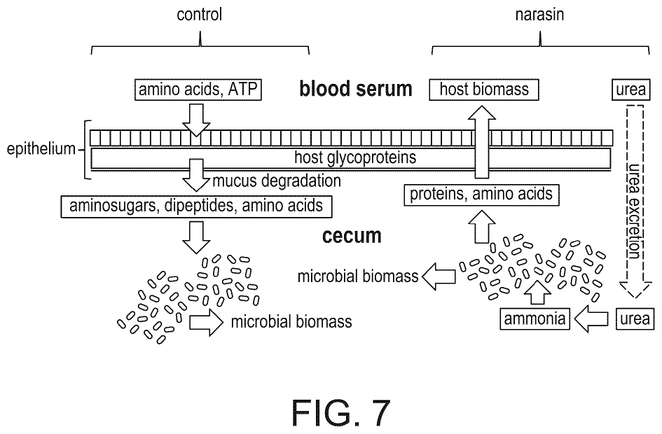

Notably, all 20 amino acids were found at lower concentrations in the cecum of narasin treated birds (FIG. 6A). Urea, on the other hand, was found at lower levels in the serum of narasin treated birds compared to controls (FIG. 6B). Thus, the abundance of several metabolites associated with altered demands or enriched functions according to the metabolic models and GSEA analyses were also found to have differential abundances in the metabolomics data. Examples include a higher concentration in the cecum of heme degradation metabolites such as bilirubin, biliverdin and others, and the enrichment of heme and tetrapyrrole biosynthesis related genes (FIG. 13B), the lower demand and higher concentration of riboflavin in the cecum of narasin treated birds (FIG. 13C), and the higher demand and lower concentration in the cecum and serum of spermidine (FIG. 13D).

Finally, the inventors investigated the correlation between the cecum and serum metabolite abundances and the overall weight gain by birds in each of the two treatments. GSEA of metabolites ranked by the strength of their correlation with weight showed that in both treatments (control and narasin) and organs (cecum and blood), the abundance of long chain fatty acids tended to be negatively correlated with weight gain. Additionally, in the serum of narasin treated birds and the cecum of control birds, metabolites from primary and secondary bile acid metabolism were positively correlated with performance. These observations, along with the lower concentration of long-chain fatty acids in the cecum of narasin treated birds, suggest that lipid metabolism and absorption play a role in AGP mechanisms as previously proposed. Interestingly, amino acids and dipeptides showed contrasting enrichment patterns between the control and narasin treatments. While both types of molecules were less abundant in the cecum of narasin treated birds, their abundance in the cecum tended to be positively correlated with growth when narasin was provided, but negatively correlated in controls (FIG. 6C). In addition, dipeptides were positively correlated with growth when measured in the serum of control birds. A similar pattern was observed for amino acids in serum for both treatments (FIG. 6D). Thus, the inventors' observations support the prediction from the metabolic models that nitrogen metabolism in the cecum was likely altered by narasin treatment and suggest that this effect might have contributed to the nitrogen balance of the host.

The gut microbiome is often viewed as a virtual organ with its own development, heritability, and metabolic, immunologic and endocrine functions. Like other organs, the microbiome plays key physiological roles and underlies different pathologies. The analogy is especially useful for predictable and relatively stable gut microbiomes such as those in the cecum of poultry in production farms. This reproducibility, aided in part by their homogeneous diets and standard rearing conditions, make chickens a good model system to understand and eventually engineer microbiome functions. As AMR concerns escalate due in part to the widespread application of AGPs in animal agriculture, understanding how the gut microbiome contributes to animal performance can point to novel strategies to maintain animal protein production while reducing the use of antibiotics.

The present inventors showed that four distinct AGPs changed the composition, gene content and AMR profile of the cecum microbiome of chickens. Several gene functions were enriched or depleted by single or multiple of the AGP treatments. Interestingly, many of the identified shifts in the abundance of gene functions were driven by the change in the proportions of a reduced set of core microbiome members. The metabolic requirements and outputs associated with the growth of those microorganisms contribute to the observed effects on performance. Using draft metabolic reconstructions for 25 core genera in the cecum microbiome the inventors estimated that the microbiome associated with the AGPs producing the largest effects on performance had fewer metabolite requirements for growth and were able to synthesize biomass from more diverse carbon and nitrogen sources. Thus, the observation that a high microbial density (˜10¹⁰-10¹¹ cfu/g) with a defined composition is maintained in the chicken cecum provides a starting point to define a direction of causality based on the impact of microbial growth and metabolism on host physiology.

One of the functions of the avian cecum is re-cycling of excreted nitrogen from the host. It has been reported that under diets low in nonessential or intact protein, urea excreted by the host is refluxed to the cecum where urea is hydrolyzed to ammonia. It was also shown that nitrogen from urea supplied directly to the cecum is re-absorbed not as ammonia but as protein, urea, and amino acids. The inventors' observations of higher levels of urea degradation genes in the cecum, lower urea levels in the serum, and higher predicted core microbiome capacity to use urea and ammonia as nitrogen sources in narasin treated birds support the hypothesis that nitrogen recycling contributes to AGP effects on performance. This is also consistent with the positive correlation between urea degradation genes and bird weight at day 35 observed in control birds. A higher turnover of urea to usable amino acids in the cecum would support protein synthesis by the host while also reducing the potential toxicity of urea in the blood.

It was recently shown that mice fed antibiotics display lower levels of host supplied nitrogen in the large intestine, which was in part mediated by lower synthesis of mucus proteins by the intestinal epithelium. Interestingly, following narasin treatment, the inventors observed a depletion of microbial genes involved in the metabolism of N-acetylneuraminate and hyaluronic acid, which are carbohydrate components of the intestinal epithelium extracellular matrix. Thus, it is possible that the cecum microbiome of narasin treated birds is less reliant on host derived carbohydrates and proteins (FIG. 6). The concentration of both amino acids and dipeptides was lower in the cecum of narasin treated birds. This observation can be explained in multiple ways: higher absorption of amino acids and dipeptides by the host, lower degradation of host proteins by the cecum microbiome, or a lower production or higher utilization of these molecules by cecum microbes. However, the positive correlation trend observed between bird weights and amino acid and dipeptide levels in the serum and the negative correlation observed in the cecum of control birds suggest that nitrogen flux from the cecum to the host may positively impact performance. In the case of narasin treated birds, amino acid levels in the serum and amino acid and dipeptide levels in the cecum correlated positively with performance. Given the predicted ability of urea and ammonia utilization as main nitrogen sources, and assuming that the lower levels of amino acids and dipeptides in the cecum are due to reduced degradation of host proteins, the positive correlation observed in the cecum may reflect a higher supply by the microbiome of usable nitrogen in birds showing the best performance.

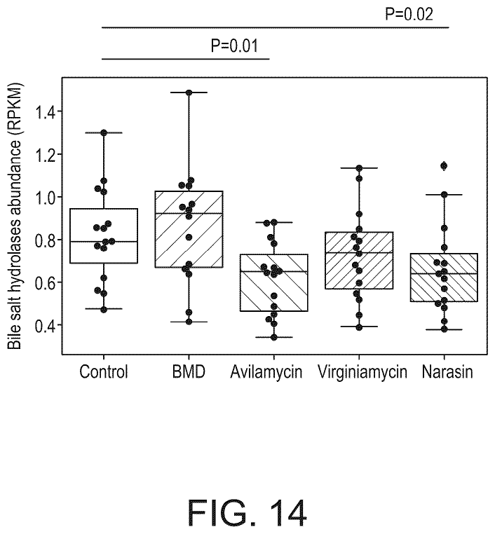

In addition to nitrogen metabolism, the results showed that the concentration of primary and secondary bile salts as well as long-chain fatty acids were altered as a function of bird weight and upon narasin treatment. Additionally, the inventors observed a trend of lower abundances of bile salt hydrolases in the AGP treatments compared to controls (FIG. 14). This is consistent with the previously proposed hypothesis that AGPs may drive performance by maintaining higher levels of active bile salts (inhibiting microbes carrying bile salt hydrolases) and thus facilitating absorption of fatty acids. Other processes of interest include the enrichment by narasin of heme and tetrapyrrole metabolism genes and the observation of higher levels of bilirubin and biliverdin in the cecum. In chickens, there is a limited capacity of the liver to excrete bilirubin, instead biliverdin produced by the catabolism of heme is the main bile pigment in bile. Thus, it has been proposed that bilirubin is a product of bacterial reduction and dehydrogenation of biliverdin. Bilirubin has antioxidant and anti-inflammatory activity which might have contributed to the higher performance of narasin treated birds. The observation of enriched tetrapyrrole metabolism, heme, siroheme, and cytochrome biosynthesis genes with narasin, further suggests that this AGP might have contributed to the synthesis and subsequent degradation of heme and related molecules directly in the cecum. Interestingly, heme degradation produces equimolar amounts of biliverdin and carbon monoxide (CO) and the Wood-Ljungdahl Pathway, which was enriched by both BMD and narasin, consumes CO and CO₂ for acetogenesis, a potential source of metabolizable energy.

The lower prevalence of microbial genes associated with the degradation of matrix polysaccharides observed with narasin may also point to decreased inflammation. On one hand, this could result from reduced mucus production which is a symptom of decreased inflammation. On the flip side, lower mucus degradation by the gut microbiome could itself alleviate the inflammatory response, as increased degradation of host glycoproteins by the gut microbiota has been associated with erosion of the mucosal barrier.

Finally, the inventors' analysis of the virginiamycin cecum microbiome showed that the treatment increased the likelihood of observing high levels of Salmonella and Escherichia carrying multiple virulence and AMR genes. Although the inventors saw a higher diversity and abundance of AMR genes after virginiamycin treatment, overall changes in AMR gene abundance were mild and in a similar direction as changes in overall gene content and taxonomic profiles. Thus, it is plausible that at the subtherapeutic dosages used in this study, the AMR profiles produced by different AGPs reflect changes in microbial population structures, rather than specific selection for resistance. The inventors note that AGP treatment sometimes resulted in lower AMR gene levels for certain AMR classes and that the types of AMR genes enriched by specific AGPs did not necessarily match the AGP class. A negative correlation between bird weight and Escherichia loads in the trachea of 7-day old antibiotic-free birds was observed (Johnson et al., Appl Environ Microbiol 84 (2018)). In the study, virginiamycin treated birds still showed increased performance compared to controls despite the much higher Enterobacteriaceae loads. So, even though inhibition of potential pathogens may contribute to AGP-mediated performance increases, it was probably not the main driver of this effect in the study.

Accordingly, growth promotion induced by different AGPs is accompanied by both common and unique cecal microbiome differences at the taxonomic, metagenomic, and metabolomic levels compared to untreated controls. Furthermore, several of these differences support mechanistic hypotheses as to how microbial activities may contribute to bird health and performance. Thus, charting the landscape of microbiome functional changes induced by antibiotics as well as other types of nutritional interventions, together with a detailed description of how the physiology of individual microbes contributes to such processes (e. g. through genome-scale metabolic modeling), should set the stage for precision microbiome engineering in both animal and human health.

### EXAMPLES

### Methods

### Clinical Study

The live animal experiment and procedures were approved by Elanco institutional animal care committee, approval number IACUC #EIAC-0773.

500 healthy Ross 708 male broilers sourced from a commercial hatchery were used in the experiment. Birds had an approximate weight of 35 to 45 g at the start of the study and received a 1× dose of Coccivac®-B52 vaccine (Merck Animal Health) within 24 hours of arrival at the study site. Birds were individually tagged with a unique identifier and were randomly assigned to one of 25 floor pens (20 animals per pen). Groups of 5 pens were assigned to each of 5 different treatments. The 5 treatments included: 1) control birds fed least-cost diets as follows: A starter diet was fed from days 0 to 14 of the study, followed by a grower diet (14-28d) and a finisher diet (28-35d). 2) Birds fed the above diets supplemented with 50 g/ton BMD. 3) Supplemented with 20 g/ton avilamycin. 4) Supplemented with 16.5 g/ton Virginiamycin. And 5) supplemented with 70 g/ton narasin. Birds were raised for 35 days in a barn on top of used litter.

Individual body weights were collected on days 0, 7, 21 and 35. Pen weights were collected on days 14 and 28. Feed weigh backs for each pen were done on days 7, 14, 21, 28 and 35 of the study. Average bird weights and feed intake per pen were used to calculate weekly values of average daily gain and feed conversion ratios. Weekly values were compared between treatments using the Mann Whitney U test. On days 7, 21 and 35 three birds per pen were randomly selected and euthanized for the collection of cecal contents and blood. Blood was collected via wing vein venipuncture, except for birds that were too small, in which cases direct heart puncture was used. In both cases, blood was collected into vacutainer tubes and centrifuged to obtain serum. Cecal samples were collected by aseptically squeezing the cecum content into a sterile tube. Cecal samples were placed on dry ice immediately after collection. All samples were stored at −80° C. until processing.

### 16S Sequencing and Analysis

Total DNA from frozen cecal content samples were extracted using the Lysis and Purity kit (Shoreline Biome, Farmington, CT) following the manufacturer's protocol. The resulting DNA was used for library preparation with the Shoreline Biome V4 16S DNA Purification and Library Prep Kit (Shoreline Biome, Farmington, CT). PCR amplification of the V4 region of the 16S rRNA gene was performed using the 515F (5′GTGGCCAGCMGCCGCGGTAA) and 806R (5′-GGACTACHVHHHTWTCTAAT) primers. The resulting amplicons were then sequenced using 2×250 bp paired-end kits on the Illumina MiSeq platform.

Paired raw reads were processed with cutadapt (v. 2.5) (Martin, M. Cutadapt 2011 17, 3, doi: 10.14806/ej. 17.1.200 (2011)) followed by DADA2 (v.1.12.1) (Callahan et al., Nat Methods 13, 581-581 (2016)) to generate a matrix of read counts per sample at the level of amplicon sequence variants (ASVs). DADA2 parameters maxN=0, truncQ=2, rm.phix=TRUE and maxEE=2 were used. The assign Taxonomy method of DADA2 was used to assign genus-level taxonomic labels to each of the ASVs based on the Silva v. 138 database (Yilmaz et al., Nucleic Acids Res 42, D643-648 (2014)). Reads assigned to the Vibrionaceae family were removed from the analysis, these correspond to a small amount (˜1% relative abundance) of Vibrio harveyii that was spiked in some of the samples. The mean sequencing depth after filtering was 43,955 reads per sample (standard deviation=12,391). The Chao, Simpson, and Shannon measures of alpha diversity were calculated from the matrix of raw counts using the scikit-bio (v. 0.5.6) python package and compared across treatments at each sampling time using the Mann-Whitney U test. The raw counts matrix was sum-normalized to generate a matrix of relative abundances of ASVs per sample. Bray-Curtis dissimilarities between all pairs of samples were calculated from the relative abundance matrix using the pdist method in the sicpy (v. 1.3.1) python package. Principal component analysis was carried out using the implementation in the scikit-learn (v. 0.21.3) python package. ANOSIM analyses between all treatment and time point combinations were carried out using the anosim method in scikit-bio.

### Shotgun Metagenomics Sequencing and Analysis

74 frozen cecal samples from day 35 birds were sent to CosmosID (Rockville, MD) for shotgun metagenomics sequencing. DNA was extracted using the ZymoBIOMICS DNA Miniprep Kit (ZymoBIOMICS, Irvine, CA) according to the manufacturers protocol. The isolated DNA was quantified by Qbit (ThermoFisher, Coon Rapids, MN). DNA libraries were prepared using the Illumina Nextera XT library preparation kit (Illumina, San Diego, CA), with a modified protocol. Libraries were then sequenced on an Illumina HiSeq platform 2×150 bp.

Reads were trimmed and Illumina adapters removed using Trimmomatic (v 0.39) (Bolger et al., Bioinformatics 30, 2114-2120 (2014)) with options SLIDINGWINDOW:5:20 LEADING:3 TRAILING:3. Host reads (Gallus gallus, GenBank assembly GCA_000002315) and spike-in reads from Vibrio harveyii (ATCC 14126) were removed using Bowtie2 (v. 2.3.5.1) (Langmead et al., Nat Methods 9, 357-359 (2012)).

Taxonomic profiling of individual reads was carried out using Kaiju (v. 1.7.3) (Menzel et al., Nat Commun 7, 11257 (2016)) with the nr_euk database. Kaiju outputs were summarized for each read as the number of bases aligned to proteins in the reference database. In cases of individual reads mapping to more than one taxonomic group, the number of bases assigned to each taxon was calculated as the alignment length (in base pairs) multiplied by the fraction of bases assigned unambiguously to said taxon out of all the taxa to which the read mapped. The numbers of assigned bases per read were used to estimate species-level relative abundances using metametamerge (v. 1.1) (Piro, V. C., et al., Microbiome 5, 101 (2017).) taking into account the average coding length per species in the nr_euk database.

Functional profiling was done by first assembling the reads from each sample into contigs using Spades (v. 3.13.1, option --meta) (Nurk, S. et al., Genome Res 27, 824-834 (2017)), with default parameters, followed by protein prediction with Prokka (v. 1.14.5) (Seemann, T. Prokka: Bioinformatics 30, 2068-2069 (2014).). Predicted protein sequences were aligned to a reference database and using Diamond (v. 0.9.30) (Buchfink, B. et al. Nat Methods 18, 366-368 (2021)) and annotated based on the function assigned to the top hit in the database. To avoid misannotations, (Plata, G. et al. Nat Chem Biol 8, 848-854 (2012)) the inventors only considered hits with at least 70% identity and 70% query coverage. The reference database was built starting from 46,181 complete bacterial genomes downloaded from the PATRIC (Davis, J. J. et al. Nucleic Acids Res 48, D606-D612 (2020)) database representing at most 10 genomes per species from ˜400 taxonomic families including those most frequently observed in broiler microbiomes. To speed up functional annotations, protein sequences within each family in the database were clustered at 90% sequence identity using CD-HIT (v. 4.8.1) (Huang, Y. et al. Bioinformatics 26, 680-682 (2010)) and the longest sequence from each cluster was kept for the diamond search.

To quantify the abundance of each function per sample, reads were mapped to the corresponding contigs using BWA (v. 0.7.17) (Li, H. & Durbin, R. Bioinformatics 25, 1754-1760 (2009)), and reads mapping to each predicted protein were counted with HTseq (v. 0.11.3) (Anders, S. et al. Bioinformatics 31, 166-169 (2015)). Read counts per RAST functional role were TPM normalized based on the Prokka protein lengths (Zhao, S. et. al. RNA 26, 903-909 (2020)) and expressed in units of mean copy numbers per genome by calculating a normalization factor such that the median abundance of a set of universal-single copy bacterial genes (Creevey, C. J. et al. PLOS One 6, e22099 (2011)) was set to 1 (Manor, O. & Borenstein, E. Genome Biology 16, 53 (2015)). A similar procedure was used to produce a copy-number normalized matrix of functional roles across samples stratified by the bacterial genera of the protein hits used to transfer functional annotations. This matrix was used to study the likely contribution of core cecum microorganisms to functional differences between samples.

ANOSIM analyses on the metagenomics derived taxonomic and functional profiles were calculated as described for the 16S data; except that the copy number-normalized abundances were used for functional profiles. Gene set enrichment analysis (GSEA) was carried out using the prerank method in the gseapy (v. 0.10.2) python package. For this, gene sets were defined based on the RAST hierarchy of functional roles parsed from PATRIC genomes. For each AGP, functional roles with mean copy numbers higher than zero in at least two samples were ranked based on the Z-score of their normalized abundances compared to controls (Equation 1) (DeGroot, M. H. Probability and Statistics. Third edn, (2002)).

$$Z = \frac{{μ}_{AGP} - {μ}_{control}}{\sqrt{\frac{{σ}_{AGP}^{2}}{{n}_{AGP}} + \frac{{σ}_{control}^{2}}{{n}_{control}}}} \qquad ( 1 )$$

where μs represent the average copy number of the roles in the respective treatment and σs represent the corresponding standard deviations. An equivalent analysis was performed by ranking functional roles with mean copy numbers higher than zero in at least three samples according to the Spearman correlation coefficient of their abundance and corresponding bird weights at day 35. The analysis was done separately for each treatment.

The core cecum microbiome was defined based on the metagenomics-based genus-level taxonomic profiles. Genera were ranked based on their mean relative abundances across samples, and the most abundant genera adding up to 90% of mean relative abundances were defined as core. The abundances of Prokka predicted genes with top diamond hits belonging to these genera were used to calculate the core microbiome contribution to functional abundances. Specifically, as the sum of the abundances of the genes with top hits in core genera. GSEA was carried out as described above for the comparison between AGPs and controls but using only functional role abundances of contributed by core genera.

### AMR Gene Abundance Analysis

AMR gene abundances per sample were determined using the AMR++ pipeline (Lakin, S. M. et al., Nucleic Acids Res 45, D574-D580 (2017)) (v. 2.0.2) starting from the raw metagenomics sequences and using the MegaRes (v. 2.0.0) database. Only genes involved in drug resistance were considered. The AMR gene counts per sample obtained from AMR++ were expressed in unites of reads per kilobase per million reads (RPKM) based on the corresponding gene lengths from the MegaRes database. RPKM values across treatments were compared using the Mann-Whitney U test.

### GSMM Reconstruction of Core Genera in the Cecum

Starting from the diamond search results (filtered at 70% identity and 70% coverage) the inventors collected the RAST gene functional roles of the top hits of predicted genes in the metagenomics assemblies. Only top hits belonging to the 25 core genera were considered. For the metabolic reconstructions the inventors focused only on functional roles that were mapped to reactions in the ModelSeed database (https://github.com/ModelSEED/ModelSEEDDatabase). The mean number of unique metabolic roles per genus based on the metagenomics data was 398 whereas the mean number of roles across fully sequenced PATRIC genomes of the same genera was 453. Thus, we used the metabolic roles from reference sequenced genomes to produce complete lists of roles for each core genus. Specifically, the inventors used SiGMoiD (Zhao, X., Plata, G. & Dixit, P. D. SiGMoiD: bioRxiv, 2020.2010.2014.338277, doi: 10.1101/2020.10.14.338277 (2021)), a recently developed statistical framework for modeling binary data, to estimate the probability that each metabolic role not present in the annotation of a genus is indeed present in said genome given its metabolic roles from the metagenomics annotation. The inventors then added the top most probable functional roles for each genus up to the mean number of metabolic roles from corresponding sequenced representatives.

Once the inventors obtained a list of metabolic roles for each core genera, the inventors relied on the mapping files from the ModelSeed database to generate draft metabolic reconstructions. A custom set of scripts was used to, for each set of roles, produce a list of corresponding metabolic reactions for a given genus. The template biomass compositions for Gram-positive and Gram-negative bacteria from ModelSeed were used as the biomass synthesis reactions of corresponding core genera.

Metabolic reconstructions were built following a similar approach to that of ModelSeed (Devoid, S. et al., Methods Mol Biol 985, 17-45 (2013); (Henry, C. S. et al., Nat Biotechnol 28, 977-982 (2010)). Gap-filling reactions were added according to the set of reaction penalties specified in the ModelSeed database, reaction bonus scores were also calculated for each model based on the presence of reactions in RAST subsystems and “scenarios” in each of the reconstructions, as previously described 75. A linear optimization problem was formulated starting from a universal metabolic network made from all reactions in the ModelSeed database. In the optimization, the inventors enforced a minimal level of biomass production (0.001 mMol/g dry weight (DW)) and minimized fluxes through reactions not present in the initial reconstruction of a specific genus, weighted by corresponding reaction scores (penalties+bonuses) (Latendresse, M., BMC Bioinformatics 15, 225 (2014)). The optimization was done on a simulated complete media; meaning that uptake of all metabolites for which transport reactions were present in the initial reconstruction were allowed.

### Flux Balance Analysis of Core Genera Metabolism

The inventors collected lists of carbon and nitrogen containing compounds with transport reactions across all 25 reconstructions. The inventors then used FBA to predict the ability of each of these compounds to serve as the main carbon or nitrogen source for growth as described previously (Plata, G., Henry, C. S. & Vitkup, D. Nature 517, 369-372, doi: 10.1038/nature13827 (2015)). Briefly, to test if a carbon source can support growth, the inventors used FBA to predict the maximum biomass synthesis rate when the total uptake fluxes through all carbon containing compounds were limited to 10 mM of carbon per gDW. Then for each tested compound, the inventors simulated maximum biomass production when that compound was not included in the above constraint. If the value obtained was at least twice the baseline the inventors considered that such compound could be used as a main carbon source. An analogous analysis was done for nitrogen sources.

The utilization potential of individual compounds was calculated as the sum of the core relative abundances of genera predicted to use that compound as a carbon or nitrogen source. Carbon and nitrogen source utilization potentials across treatments were compared using the Mann Whitney U test.

To calculate the metabolic demand for a given sample the inventors did the following. First, the inventors calculated an essentiality score for each metabolite with transport reactions for each of the 25 genera. The essentiality score for a given metabolite was defined as the fraction of in silico media conditions in which the metabolite was essential for biomass synthesis. To simulate media conditions for a given reconstruction, the inventors closed all uptake reactions and then randomly opened a random subset of uptake reactions by sampling from a binomial distribution with p=0.9. If the random media composition thus defined supported biomass synthesis, the inventors tested the essentiality of every metabolite with opened uptakes. For this, the uptake reaction of the metabolite was closed, and the feasibility of biomass synthesis was evaluated with FBA. If the model was unable to simulate biomass synthesis after closing the uptake of the metabolite, the metabolite was deemed essential for that condition. Altogether, a total of 1000 viable random media were considered for each genus, and the essentiality score of each metabolite was calculated relative to the number of media in which the metabolite was present. Second, the essentiality scores for each genus were averaged across metabolites and multiplied by the core abundance of the genus. Third, the inventors summed the product of core relative abundances and average essentiality scores across core genera. For individual metabolites, the metabolic demand was calculated as the sum across core genera of the essentiality scores for the metabolite multiplied by core relative abundances.

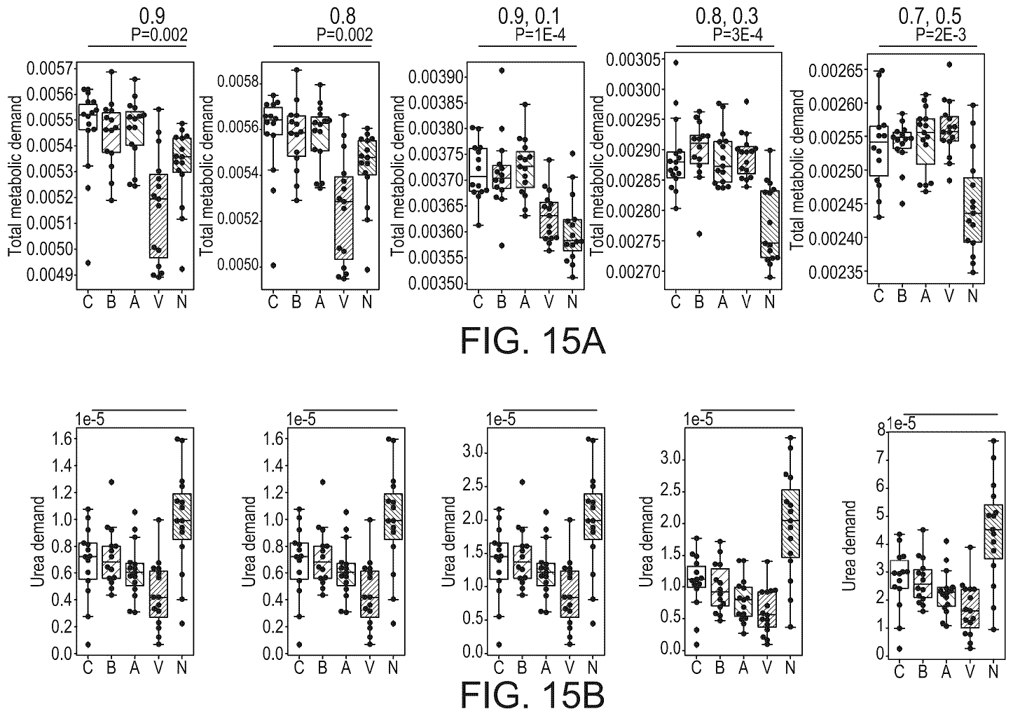

The 90% probability for external metabolites used to simulate random media was used to account for differences in metabolite essentiality across environmental conditions. Similar results were obtained at lower metabolite probabilities and when, in addition to metabolites with transporters, reactions for the direct uptake of metabolites without transporters were also included with various probabilities (FIG. 15). This was done to account for possible transport reactions missing in the metagenomics annotation.

### Untargeted Metabolomics

15 cecal samples and 10 serum samples from day 35 birds were sent to metabolon (Morrisville, NC) for untargeted metabolomics analysis. These numbers of samples were sent for both the control and narasin treatments. Serum samples were selected sequentially, that is, without knowledge of bird performance or results from other analyses.

Samples were prepared using the automated MicroLab STAR® system from Hamilton Company. Samples were extracted with methanol under vigorous shaking for 2 min (Glen Mills GenoGrinder 2000) to precipitate protein and dissociate small molecules bound to protein or trapped in the precipitated protein matrix, followed by centrifugation to recover chemically diverse metabolites. The resulting extract was divided into four fractions: two for analysis by two separate reverse phase (RP)/Ultrahigh Performance Liquid Chromatography-Tandem Mass Spectroscopy (UPLC-MS/MS) methods using positive ion mode electrospray ionization (ESI), one for analysis by RP/UPLC-MS/MS using negative ion mode ESI, and one for analysis by HILIC/UPLC-MS/MS using negative ion mode ESI. Samples were placed briefly on a TurboVap® (Zymark) to remove the organic solvent. The sample extracts were stored overnight under nitrogen before preparation for analysis.

The sample extract was dried and reconstituted in solvents compatible to each of the four methods. One aliquot was analyzed using acidic positive ion conditions, chromatographically optimized for more hydrophilic compounds. In this method, the extract is gradient-eluted from a C18 column (Waters UPLC BEH C18-2.1×100 mm, 1.7 μm) using water and methanol, containing 0.05% perfluoropentanoic acid (PFPA) and 0.1% formic acid (FA). A second aliquot was also analyzed using acidic positive ion conditions but was chromatographically optimized for more hydrophobic compounds. In this method, the extract is gradient eluted from the C18 column using methanol, acetonitrile, water, 0.05% PFPA and 0.01% FA, and is operated at an overall higher organic content. A third aliquot was analyzed using basic negative ion optimized conditions using a separate dedicated C18 column. The basic extracts were gradient-eluted from the column using methanol and water, and with 6.5 mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10 mM Ammonium Formate, pH 10.8. The MS analysis alternates between MS and data-dependent MSⁿ scans using dynamic exclusion. The scan range varies slightly between methods, but covers approximately 70-1000 m/z.

UPLC-MS/MS was done using a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution.

Raw data was extracted, peak-identified, and QC processed using Metabolon's hardware and software. Compounds were identified by comparison to library entries of purified standards. The library, based on authenticated standards, contains the retention time/index (RI), mass to charge ratio (m/z), and chromatographic data (including MS/MS spectral data) on all molecules present in the library. Biochemical identifications were based on three criteria: retention index within a narrow RI window of the proposed identification, accurate mass match to the library+/−10 ppm, and the MS/MS forward and reverse scores. MS/MS scores are based on a comparison of the ions present in the experimental spectrum to ions present in the library entry spectrum.

Peaks were quantified as area-under-the-curve (AUC) detector ion counts. For each identified metabolite, AUC values were rescaled by dividing by the median AUC value across all samples. Any missing values after re-scaling were imputed with the minimum value observed for that compound across samples. Metabolite abundances between treatments in each organ were compared using Welch's t tests on the log-transformed scaled and imputed data. Correlations between metabolite levels and corresponding bird weights at day 35 were calculated using the Pearson correlation of the log-transformed data.

A classification of metabolites into pathways and super-pathways was used for running GSEA based on metabolites ranked by their Welch's t score between treatments and by their correlation with weight. A minimum set size of 3 metabolites per set was used.

### Data Availability

Raw 16S and shotgun metagenomics sequences were deposited in the SRA database under accession number PRJNA751585. Reactions in the draft metabolic reconstructions analyzed for the 25 core genera are available from (https://github.com/platyias/BroilerCoreModels).

1. A method of selecting an antimicrobial growth promoter (AGP) treatment for a domesticated organism in order to promote a healthy microbiome, the method comprising:
a) providing a plurality of test organisms and a control organism,
b) administering a regular regimen of a different AGP to each test organism for up to about two months,
c) collecting DNA from the intestinal content of the test organisms and of the control organism,
d) sequencing the DNA of the intestinal content of at least one test organism and of the control organism to obtain a taxonomic profile, and, optionally, a functional profile, thereby defining core intestinal microbiome of the test organism(s) and of the control organism,
e) comparing the core intestinal microbiome of the test organism(s) and of the control organism, thereby determining antimicrobial resistance (AMR) gene abundance of the test organism(s), and determining which test organism has the most healthy microbiome thereby determining which AGP provides the most healthy microbiome, and
f) selecting the AGP which provides the most healthy microbiome, wherein the domesticated organism, test organism and control organisms are of the same species.

2. The method of claim 1 wherein the organism is selected from the group consisting of birds, pigs and cows.

3. The method of claim 1 wherein the bird is a chicken.

4. The method of claim 1 wherein the AGP is selected from the group consisting of bacitracin methylene disalicylate (BMD), avilamycin, virginiamycin and narasin.

5. The method of claim 1 wherein the intestinal content is collected from the cecum.

6. The method of claim 1 wherein the administering a regular regimen of a different AGP to each test organism is for about 35 days.

7. The method of claim 1 wherein the administering a regular regimen of a different AGP to each test organism is for about 14 days.

8. The method according to claim 1 wherein an AGP which provides a healthy microbiome is a microbiome which is able to synthesize biomass from more diverse carbon and nitrogen sources, and/or has fewer metabolite requirements for microbial growth.

9. The method according to claim 1 wherein an AGP which provides a healthy microbiome is associated with an increase in at least one of: metabolism of aromatic amino acids, propanediol utilization, molybdenum-containing cofactor synthesis, CO₂ fixation via the Wood-Ljungdahl pathway, protein synthesis, biotin synthesis, methionine metabolism, glycolysis, sporulation, soprenoid synthesis, betaine biosynthesis, pyridoxine biosynthesis, stress response, bacterial secretion systems, antimicrobial resistance, biosynthesis of Gram-negative cell wall components, tryptophan synthesis, urea degradation, heme/tetrapyrrole biosynthesis, bilirubin, biliverdin and xylose utilization.

10. The method according to claim 1 wherein an AGP which provides a healthy microbiome is associated with at least one of: decreased metabolites implicated in amino acid metabolism, decreased long-chain fatty acids, increased levels of metabolites from primary and secondary bile acid metabolism, increased levels of metabolites related to hemoglobin and porphyrin metabolism, increased levels of metabolites related to fructose and galactose metabolism, increased levels of tocopherol, inositol, riboflavin, nicotinate and/or nicotinamide metabolism.

11. The method according to claim 1 wherein an AGP which provides a healthy microbiome is associated with decreased levels of microbial genes associated with degradation of matrix polysaccharides and BSH genes, and/or higher levels of riboflavin synthase genes.

12. A method of treating a domesticated organism with an antimicrobial growth promoter (AGP) to promote a healthy microbiome, the method comprising:
a) providing a plurality of test organisms and a control organism,
b) administering a regular regimen of a different AGP to each test organism for up to about two months,
c) collecting DNA from the intestinal content of the test organisms and of the control organism,
d) sequencing the DNA of the intestinal content of test organisms and of the control organism to obtain a taxonomic profile, and, optionally, a functional profile, thereby defining core intestinal microbiomes of the test organisms and of the control organism,
e) comparing the core intestinal microbiomes of the test organisms and of the control organism, thereby determining antimicrobial resistance (AMR) gene abundance of the test organisms, and determining which test organism has the most healthy microbiome thereby determining which AGP provides the most healthy microbiome, and
f) treating the domesticated organism with the AGP which provides the most healthy microbiome, wherein the domesticated organism, test organism and control organisms are of the same species.
